(19) United States
(12) Patent Application Publication
(10) Pub. No.: US 2015/0201867 A1
(43) Pub. Date: Jul. 23, 2015

(54) ELECTRONIC FREE-SPACE MOTION MONITORING AND ASSESSMENTS

(71) Applicants: The Charlotte Mecklenburg Hospital Authority d/b/a Carolinas Healthcare System, Charlotte, NC (US); The University of North Carolina at Charlotte, Charlotte, NC (US)

(72) Inventors: Richard D. Peindl, Charlotte, NC (US); Naiquan Zheng, Charlotte, NC (US)

(21) Appl. No.: 14/600,526

(22) Filed: Jan. 20, 2015

Related U.S. Application Data
(60) Provisional application No. 61/929,560, filed on Jan. 21, 2014.

(51) Int. Cl. A61B 5/11; A61B 5/00

(52) U.S. Cl. CPC A61B 5/1118; A61B 5/0022; A61B 5/1123; A61B 5/1117; A61B 5/4848; A61B 5/7282; A61B 2503/40; A61B 2505/07

(57) ABSTRACT

The systems, methods and computer program products evaluates motion data obtained from an IMU of different respective users to identify when a physical activity occurs and, what physical activity occurs using waveform template matching to corresponding user waveforms with activity signatures and optionally how well the physical activity was performed relative to a reference population or a baseline performance of the same physical activity by the user.

### RELATED APPLICATIONS

This application claims the benefit of and priority to U.S. Provisional Patent Application Ser. No. 61/929,560, filed Jan. 21, 2014, the contents of which are hereby incorporated by reference as if recited in full herein.

### RESERVATION OF COPYRIGHT

A portion of the disclosure of this patent document contains material which is subject to copyright protection. The copyright owner, The Charlotte-Mecklenburg Hospital Authority, doing business as “Carolinas HealthCare System”, Charlotte, N.C., has no objection to the reproduction by anyone of the patent document or the patent disclosure, as it appears in the Patent and Trademark Office patent file or records, but otherwise reserves all copyright rights whatsoever.

### FIELD OF THE INVENTION

The invention relates to systems, methods and computer programs used to provide activity monitoring.

### BACKGROUND

In the past, researchers have proposed different systems for identifying motion including systems with passive infrared markers and cameras and systems that employ active markers. Despite the foregoing, there remains a need for systems that can provide motion and/or activity monitoring using active sensors that are computationally efficient and reliable without requiring cameras or other external monitoring devices in the local environment. The need is particularly great for cost-effective, reliable and easy to use systems for monitoring activities of daily living (ADL) in free space.

### SUMMARY OF EMBODIMENTS OF THE INVENTION

Embodiments of the invention provide systems, methods and computer program products that electronically evaluate motion data obtained from different respective users, typically for the purpose of activity detection and identification and, optionally, also activity performance evaluation. The identifications and/or evaluations functions can be carried out by the use of template matching, which may include Boolean template matching techniques, where an individual's multi-channel sensor output data are compared with predetermined activity (reference) waveform templates.

Embodiments of the invention may also and optionally determine how well the detected and identified physical activity was performed relative to a reference population or a baseline performance of the same physical activity by the same individual. The evaluation can be at one time period or over a plurality of different intervals.

Embodiments of the invention employ Boolean comparisons of digital waveforms output from multi-channel IMUs to waveform templates to delineate specific variations in the manner in which a particular ADL may be performed.

Embodiments of the invention are directed to methods of identifying a physical action and/or activity of a user in a free space environment. The methods include: electronically obtaining digital data of physical activity and/or action of a user in free space from an inertial measurement unit (IMU) that includes a three axis accelerometer, a three axis gyroscope and a magnetometer. The IMU can be held proximate an upper torso of the user so as to be able to detect motion along and about a body axis coordinate system with three orthogonal axes, an up-down axis, a side-to-side axis and a front-to-back axis and provide orientation data of the body axis system with a global coordinate system. The method can also include electronically generating user waveforms of angular velocities and accelerations in the respective body axis system orthogonal axes over a first concurrent time period associated with a physical action and/or activity using the data obtained from the IMU; electronically generating user waveforms of at least vertical acceleration in the global coordinate system in the first concurrent time period using the orientation data with acceleration data obtained from the IMU; electronically comparing one or more waveform templates of angular velocities and/or accelerations associated with different defined actions and/or activities with one or more of the generated user waveforms; and electronically identifying physical actions and/or activities of the user based on one or more waveform template matches from the electronic comparison without requiring a camera or other motion tracking systems in the environment.

The obtaining step can be carried out while the user performs random activities in free space without requiring a camera or other motion tracking systems in the environment.

The obtaining step can be carried out while the user performs assigned physical tasks or actions in free space without requiring a camera or other motion tracking systems in the environment.

The predefined waveform templates can and/or include waveform templates of angular velocities and accelerations for specific defined activities comprising activity signatures from different users generated by calculating a mean or average of the activity signatures from the different users using IMU data obtained during each different user's performance of each specific defined activity.

The method can also include electronically evaluating performance of the identified physical action and/or activity of the user based on error thresholds of matches from the IMU waveform(s) data to template(s) based on the comparison(s) without requiring a camera or other motion tracking systems in the environment.

The IMU can be a single IMU per a respective user/patient. The identification can be carried out using the obtained data or motion data plus orientation data obtained only from the single IMU.

The generated user waveforms can include eight different waveforms over a concurrent time period from a respective eight data streams from the IMU. The electronic identification can be carried out by comparing activity signatures in less than eight of the different waveforms, each template having one or more activity signatures for a defined action and/or activity.

The generated user waveforms can include eight different waveforms over a concurrent time period. The electronic identification can be carried out by comparing activity signatures of between one and four of the different waveforms over a concurrent time period with waveform templates, each waveform template having one or more activity signatures for a defined action and/or activity.

The generated user waveforms of angular velocities and accelerations with respect to the body axis system and corresponding waveform templates can exclude gravity.

The action or activity identification and performance evaluation can include matching a primary waveform template and a secondary waveform template associated with a defined physical action or activity.

The method can include accepting user input to select one or more parameters of a reference population associated with waveform templates of angular velocities and accelerations for specific defined activities comprising activity signatures from different users during performance of each specific defined activity. The method can include electronically generating a custom set of waveform templates based on the input to thereby allow different sets or categories of waveform templates for different users and/or different sets of users.

The identifying can include calculating an error threshold of at least one waveform template to at least one generated user waveform with an activity signature. If the error threshold is below a defined value, then the physical action or activity can be identified as the action or activity defined by the physical action or activity associated with a respective at least one waveform template.

The identifying can include electronically calculating an error threshold of a first waveform template to at least one generated user waveform with an activity signature. If the error threshold is above a defined value, the method may include electronically comparing a second different selected waveform template to the at least one generated user waveform with the activity signature.

The comparing can include employing Boolean logic with different waveform templates using a defined pair or series of waveform templates that can be used to distinguish a physical action or activity and/or characterize how a physical action or activity is carried out.

The different waveform templates can include a first waveform template of vertical acceleration in an up-down axis and a second waveform template of angular velocity in a side-side axis. The first and second waveform templates can be used to identify a stand to sit or sit to stand physical action.

The method can include providing at least one database of waveform templates, each with one or more activity signatures associated with defined physical actions and/or activities for the comparing and identifying steps, the database can include one or more waveform templates for each of a plurality of the following: step up or step down, walk, sit to stand, stand to sit, reach, pick an object up, bend, stair climbing, stair descent, bed-chair transfer, and transfer from chair to toilet.

The method can include electronically identifying improvement or increasing kinematic impairment of the user by comparing generated user waveforms with activity signatures of the same physical activity or action at different times.

The method can include electronically assessing mobility status of a respective user by comparing different generated user waveform profiles with respective mean or average calculated waveform templates of the same physical action or activity generated using a reference population or subpopulation.

The method can include electronically evaluating a standardized physical activity and/or action test to assess a respective user's stability or fall risk using the generated user waveforms and the waveform templates. The evaluation of the test can include evaluating a plurality of defined actions associated with a user undergoing the test that comprises at least one sit to stand and/or stand to sit physical action.

The method can also include electronically evaluating how well a user is performing the identified action or activity by comparing maxima and/or minimas of an activity signature or signatures in one or more of the generated user waveforms to corresponding activity signatures in one or more waveform templates from a reference population.

The method can include electronically evaluating physical impairments, decreasing kinematic ability or improved kinematic ability from a drug, implant, prosthetic or orthotic of the user, using at least one of the generated user waveforms at one time and/or over time.

The waveform templates can include baseline waveforms of activity signatures of the user generated by monitoring the user when performing defined actions or activities.

The waveform templates can include activity signatures generated by calculating a mean or average of activity signatures from different users obtained during performance of each specific defined activity or action.

The evaluating can be carried out to evaluate influence or efficacy of a drug to allow a physician to proactively monitor for drug effects and/or to titrate a prescribed dose for a user.

The method can include electronically generating one or more scores of one or more physical activities or actions to assess fall risk and/or how normal or abnormal the physical activity of action was performed by a user.

Other embodiments are directed to methods for activity monitoring of users using a computer network. The methods include providing a web-based service that provides an activity monitoring analysis module using at least one server that evaluates data obtained from a multi-channel IMU of different respective users, the IMU having at least eight data streams output to the web-based service including three accelerometer data streams, three angular velocity data streams and three magnetometer data streams. The web-based activity monitoring analysis module can be configured to identify a physical activity and/or action of respective users by comparing waveform templates in a database or electronic library to user waveforms with activity signatures associated with physical actions or activities of respective users generated from the data from respective IMUs without requiring a camera or other motion tracking systems in a user environment.

The activity monitoring analysis module can be configured to evaluate how well the identified physical activity or action was performed relative to a reference population or relative to a baseline performance of the same physical activity or action.

The activity monitoring analysis module can be configured to identify the physical action or activity using template matching with a plurality of waveform templates from the database or library that have defined associated activity signatures to identify what physical activity is performed by a respective user using between 1-4 waveform templates.

The data streams can be used to generate a waveform extract of eight different user waveforms over a concurrent time period, wherein the identification is carried out by comparing activity signatures of between one and four of the different user waveforms over a concurrent time period with one or more activity signatures in one or more of the waveform templates. The waveform templates for some or all defined physical actions or activities can include waveform templates of velocities and accelerations in a respective body axis system and a waveform template of vertical acceleration in a global coordinate system.

The eight data streams can include user waveforms of angular velocities and accelerations in a respective body axis system orthogonal axes over a first concurrent time period associated with physical actions and/or activities, and a waveform of vertical acceleration in a global coordinate system using orientation data with acceleration and angular velocity data in the body axis system obtained from the IMU.

Other embodiments are directed to monitoring systems that include at least one web server in communication with a global computer network configured to provide a web-based service that hosts a monitoring analysis module that evaluates motion data obtained from an IMU comprising a three axis accelerometer, a three axis gyroscope and a magnetometer associated with respective different respective users to identify a physical activity carried out by respective users using data only from the IMU and a library or database of waveform templates without requiring a camera or other motion tracking systems in a free space environment.

The monitoring analysis module can be configured to identify how the physical activity was carried out using Boolean review with a defined hierarchy of selected waveform templates to compare to user waveforms generated from data from the IMU.

The monitoring analysis module can optionally evaluate how well the physical activity was performed using the waveform templates generated relative to a reference population or a baseline performance by the user of the same physical activity.

The analysis module can be configured to obtain digital data of random physical activities and/or actions of respective users in free space from a single IMU for each user, the IMU held proximate an upper torso of the user so as to be able to detect motion along and about a body axis coordinate system with three orthogonal axes, an up-down axis, a side-to-side axis and a front-to-back axis and provide orientation data of the body axis system with a global coordinate system. The analysis module can be configured to (a) generate user waveforms of angular velocities and accelerations in the respective body axis system orthogonal axes over a first concurrent time period associated with physical actions and/or activities using the data obtained from the IMU; and (b) generate user waveforms of acceleration in the global coordinate system in the first concurrent time period using the orientation data with acceleration and angular velocity data in the body axis system obtained from the IMU.

The library or database of waveform templates can include waveform templates of angular velocities and/or accelerations with activity signatures associated with defined different actions or activities based on performance of the defined different actions or activities by different users with respective IMUs.

Other embodiments are directed to computer program products, the computer program products include a non-transitory (tangible) computer readable storage medium having computer readable program code. The computer-readable program code includes computer readable program code configured to carry out the methods described herein.

Yet other embodiments are directed to systems that include at least one processor comprising computer program code that, when executed, causes the processor to carry out some or all of the operations of the methods described herein.

As will be appreciated by those of skill in the art in light of the above discussion, the present invention may be embodied as methods, systems and/or computer program products or combinations of same. In addition, it is noted that aspects of the invention described with respect to one embodiment, may be incorporated in a different embodiment although not specifically described relative thereto. That is, all embodiments and/or features of any embodiment can be combined in any way and/or combination for any number of desired activities and/or any degree of activity performance complexity or variability. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic illustration of a free space monitoring system according to embodiments of the present invention.

FIGS. 2A-2C are graphs of concurrent waveform profiles associated with body system instantaneous curvilinear accelerations (m/sec²) v. time (seconds) according to embodiments of the present invention.

FIGS. 2D and 2E are graphs of concurrent waveform profiles associated with earth-centered instantaneous horizontal and vertical accelerations (m/sec²) v. time (seconds) according to embodiments of the present invention.

FIGS. 3A-3C are graphs of concurrent waveform profiles of body system instantaneous angular velocities (rad/seconds) v. time (seconds) according to embodiments of the present invention.

FIGS. 4A and 4B are graphs of vertical acceleration and y-axis angular velocity data demonstrating Boolean template matching for the purpose of identifying similar presenting physical activities (e.g., sit-to-stand v. turn-and-sit) according to embodiments of the present invention.

FIG. 5A and FIG. 5B are schematic illustrations of Boolean template matching for identifying physical activity and activity performance evaluation according to embodiments of the present invention.

FIG. 6 is a schematic illustration of a user-selectable input for generating on-the-fly or custom, typically population-based, waveform templates using one or more databases of waveform templates having one or more physical activity waveform signatures according to embodiments of the present invention.

FIG. 7A is front view of an example of an IMU and wearable support device according to embodiments of the present invention.

FIG. 7B is a front view of another example of an IMU and wearable support device according to embodiments of the present invention.

FIG. 7C illustrates an implantable configuration of the IMU according to embodiments of the present invention.

FIG. 7D illustrates an adhesively configured configuration of the IMU according to embodiments of the present invention.

FIG. 7E is a side view of another example of an IMU and wearable support device for an animal according to embodiments of the present invention.

FIGS. 7F-7H are schematic illustrations of a body-based coordinate axis system with respect to the onboard IMU and FIG. 7H also illustrates an earth-centered coordinate axis system according to embodiments of the present invention.

FIG. 8A is a tri-axial acceleration (m/s²) v. time (seconds) graph of motion of a patient during an actual fall.

FIG. 8B is a tri-axial acceleration (m/s²) v. time (seconds) graph of motion of a simulated fall by a volunteer.

FIG. 9 is a vertical acceleration (i.e. with respect to an earth-centered coordinate system) graph of the torso as monitored during an EATUG test illustrating an irregular gait pattern according to embodiments of the present invention.

FIG. 10 is a flow chart of exemplary operations/steps that can be used to carry out embodiments of the present invention.

FIG. 11 is a schematic illustration of a data processing system according to embodiments of the present invention.

FIG. 12A is a graph of the body/torso coordinate system curvilinear accelerations along a moving IMU's three orthogonal axes which shows motion sensor data for a representative electronically augmented standardized fall-risk test, (e.g., an Electronically Augmented Timed Up and Go (EATUG) test), while FIG. 12B shows angular velocities about the same body coordinate system three moving axes for the same EATUG test.

FIG. 13A/13B are graphs of torso angular velocity about the body system x-axis for two types of turns according to embodiments of the present invention: FIG. 13A reversing turn while walking and FIG. 13B reversing turn while sitting down. The bold black curves represent the average templates for activity signatures obtained from three different individuals in three separate EATUG tests.

FIG. 14 is a composite graph of two different waveform (turn) templates and the body x-axis angular velocity waveform signatures for three different individuals performing an EATUG test. In the figure, the “walking turn” waveform template is selected for matching with the multiple activity waveform signatures according to embodiments of the present invention.

FIG. 15 is an x-axis composite graph as in FIG. 14 with the turn-to-sit template selected for matching according to embodiments of the present invention.

FIG. 16A is a y-axis (torso flexion/extension) graph of angular velocity waveform signatures with an associated template (rad/sec) of sit-to-stand motions and FIG. 16B represents y-axis angular velocity waveform signatures with an associated template (rad/sec) of stand-to-sit motions according to embodiments of the present invention. Each graph has activity waveform signatures from three different individuals with the bolder line representing the mean curve or waveform signature for those three individuals.

FIG. 17 is a y-axis composite graph with the sit-to-stand template selected for matching according to embodiments of the present invention.

### DETAILED DESCRIPTION OF EMBODIMENTS OF THE INVENTION

The present invention will now be described more fully hereinafter with reference to the accompanying figures, in which preferred embodiments of the invention are shown. This invention may, however, be embodied in many different forms and should not be construed as limited to the embodiments set forth herein.

Like numbers refer to like elements throughout. In the figures, layers, regions and/or components may be exaggerated for clarity. Broken lines illustrate optional features or operations unless specified otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the data or information in use or operation in addition to the orientation depicted in the figures. For example, if data in a window view of the system in the figures is inverted, elements described as “under” or “beneath” other elements or features would then be oriented “over” the other elements or features. Thus, the exemplary term “under” can encompass both an orientation of over and under. The display view may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

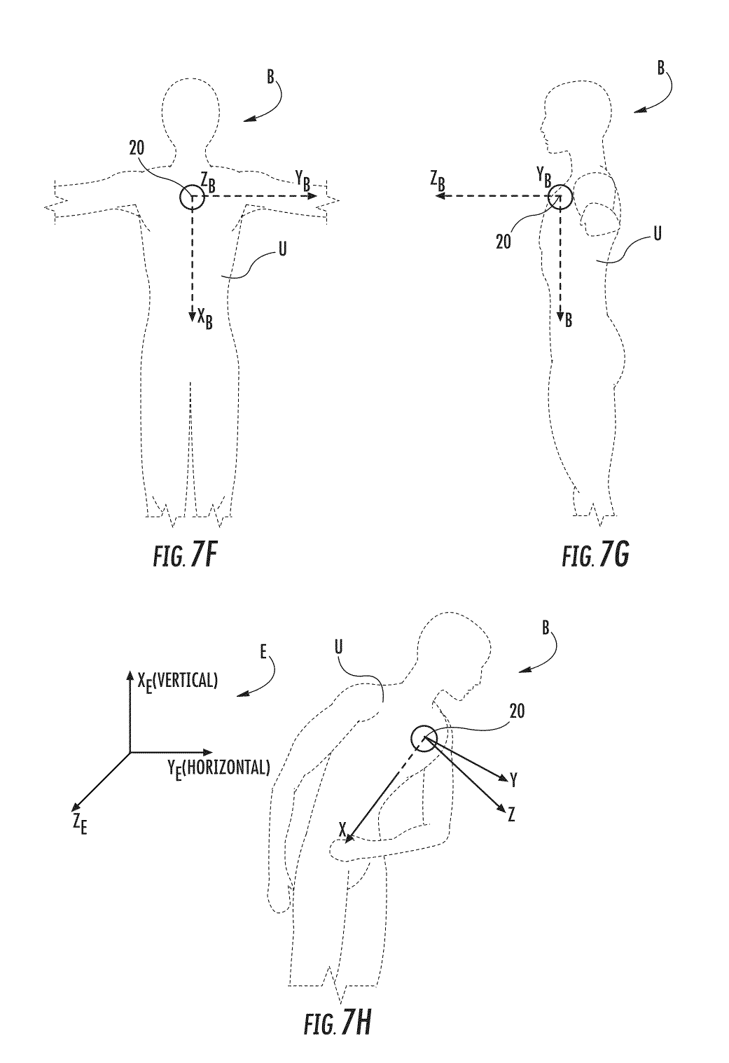

Embodiments of the invention employ two three axis coordinate systems: the IMU axis system, which is referred to as the body (or torso) coordinate system (“B”, FIG. 7H), and an earth-centered or global coordinate system (“E”, FIG. 7H), where vertical is with respect to the earth's center and horizontal can be omnidirectional in a plane orthogonal to the vertical axis. Although certain movement directions have been associated with a defined coordinate axis, the noted axis for a respective body system movement description, e.g., up or caudad and down or cephalad (x_{B}), side-to-side or medial-lateral (y_{B}) and front-back or anterior-posterior (z_{B}) movement, is by way of example for defining instantaneous accelerations or velocities in the body system (i.e., fixed to the upper torso). Terms like vertical and horizontal, can sometimes align with the body (torso) axis system, but always, when used with the earth-centered system, refer to the vertical axis and an associated orthogonal horizontal axis which may be in any horizontal direction (i.e. omnidirectional). The concept of a vertical, i.e. gravitational (earth-based), axis can be important in associating the orientation of the body-fixed coordinate system with respect to the earth-centered system, e.g., determining whether the body coordinate system is generally upright or supine or recumbent while performing an activity.

As used herein, the term “inertial measurement unit” (IMU) refers to a relatively compact or small device or sensor package that can be worn by a user. Preferred embodiments of the invention employ at least one (typically only one) IMU that has at least 9 degrees of freedom (DOF) using a tri-axis accelerometer, a tri-axis gyroscope and a tri-axis magnetometer or similar global positioning-related device providing orientation data. To be clear, there are other sensor packages that might be identified as IMUs, but do not have at least nine degrees of freedom (DOF). For example, an IMU with a single uniaxial accelerometer would be considered to be a one DOF IMU. An IMU with only a triaxial accelerometer is a 3 DOF IMU. An IMU with a triaxial accelerometer and a triaxial gyroscope is an IMU with 6 DOF. An IMU with 9 DOF includes a triaxial accelerometer, a triaxial gyroscope and triaxial magnetometer(s).

The IMU can be configured to allow sensor fusion by processing using Kalman filtering to generate orientation quaternion data. As is known to those of skill in the art, orientation quaternion data is a four digit number associated with a rotation matrix that allows calculation of vertical and horizontal acceleration at the same time. IMUs with greater than 9 DOF include additional sensors like temperature sensors for temperature compensation of circuit components/drift or barometric sensors (with temperature sensors) to determine height above the ground. See, e.g., Sabatini, A. M. Quaternion-based extended Kalman filter for determining orientation by inertial and magnetic sensing. IEEE Trans. Biomed. Eng. 2006, 53, 1346-1356; Yun et al., A simplified quaternion-based algorithm for orientation estimation from earth gravity and magnetic field measurements. IEEE Trans. Instrum. Meas. 2008, 57, 638-650; and Lee et al., A fast quaternion-based orientation optimizer via virtual rotation for human motion tracking. IEEE Trans. Biomed. Eng. 2009, 56, 1574-1582. The contents of these documents are hereby incorporated by reference as if recited in full herein.

For activity monitoring, as will be discussed below, it is preferred to use a single IMU and that the single IMU be an IMU with nine or more degrees of freedom strategically positioned to be sensitive to detect motions of ADL in a minimally obtrusive application to promote user/clinical acceptance. Where a single IMU is used, as will be discussed below it may be held snugly against skin of a user over a top portion of a sternum, but other locations and configurations are possible.

The nine DOF or higher IMU can have multiple output channels of motion data, typically the number of active sensor channels is commensurate with the number of DOF. In some embodiments, the IMU is configured to operate generating at least eight data streams; six directly from the IMU body axis system (B, FIGS. 7D-7F) from the accelerometers and the gyroscopes and two that are associated with the earth-centered or “global coordinate” axis system (E, FIG. 7F), typically calculated “on-the fly” or as the motion data is generated and/or detected from acceleration and orientation quaternion data.

The accelerometers measure, typically in m/s², the total inertial acceleration acting on the sensor. This inertial acceleration includes both linear accelerations along each of the three (orthogonal) sensor axes, a gravitational force component along each axis and possible centripetal or coriolis accelerations caused by rotations of the (IMU) sensor package. The gyroscopes measure the angular velocity of the sensor in rad/s. Because of the microelectromechanical systems (MEMS) construction nature of the compact or miniaturized IMU sensor packages, gyroscopic measurements can also at times exhibit “noisy” signals caused by abrupt accelerations of the sensor package. As is known, the signals measured by the sensors can be modeled as follows: the accelerometer measurement vector, at, at time, t, is given by:

at=at+gt+μa,t Equation (1)

where at is the linear/centripetal/coriolis acceleration component due to sensor motion, gt is the component due to the Earth's gravitational force and μa,t is a noise and/or sensor drift term. Similarly, the gyroscope signal, ωt, is given by:

ωt=wt+μw,t Equation (2)

where wt is the angular rotation and μw, t is a noise/drift term. Equations 1 and 2 are by way of example only.

It is contemplated that the at least 9 DOF IMU configuration can include (typically customized by manufacturer or design) onboard circuitry and sensor data fusion software algorithms to carry out the Kalman filters for calculation and data output of sensor package instantaneous orientation (i.e., with respect to an earth-centered system) in addition to acceleration and angular velocity data. Orientation data is usually presented in terms of orientation quaternions which are a shorthand representation for rotation transformation matrices.

In the past, IMUs have typically been used for purposes of motion tracking or “dead-reckoning” with IMU-specific navigation equations which include IMU calibration variables and sensor performance characteristics algorithms used with the IMU data output in order to reduce or minimize effects of sensor drift and noise characteristic errors, to minimize centripetal and coriolis acceleration effects on motion tracking and finally to calculate instantaneous position, velocity and orientation data with respect to a global coordinate system. Advantageously, navigation equations are not required for (and indeed are preferably not used for) activity identification or even performance evaluation with the local body system activity monitoring, which is in contrast to 3-dimensional motion tracking in a global coordinate system.

Generally stated, when doing activity monitoring with a single IMU having at least 9 DOF, the systems, methods, circuits and computer programs can be configured to consider two general perspectives: the body axis system fixed (typically affixed to the torso) “B” and the earth-centered axis system “E” that defines vertical and horizontal accelerations (FIG. 7H). As previously noted, the IMU gives acceleration and angular velocity data in the body axis system, with a respective six different data streams from six sensor channels. The IMU can also provide magnetometer data in x, y and z for orientation data over a respective three additional sensor channels. “Perspective in the body axis system” refers to actions like when a user is bending over, the systems/methods can discriminate if he/she is tilting to the right and rotating to the left or is the user picking up an object with a right or left hand. These perspectives can be more easily interpreted than rotations and accelerations converted to a global or inertial xyz-system where angular velocities and accelerations in three directions simultaneously would need to be considered to get a general perspective or sense of how a body is moving. With regard to the earth-centered system, embodiments of the motion detection and identification protocols are generally independent of what direction things accelerate in, in the horizontal plane. However, vertical acceleration can provide much more relevant information because the musculoskeletal system has to work against gravity—how well it works against gravity (speed, efficiency, etc) can be used to assess how well a user can perform an activity. Thus, gravity may be excluded from the body axis system accelerations for activity detection and identification, but may be used to generate information about the effects of gravity and how the individual works (i.e. in this case, accelerates) against gravity. If orientation quaternions are used to convert all accelerations and angular velocities into a global coordinate system, important perspectives may be lost and may require more intensive computations with navigation equations, sensor drift, bias and all the computational issues thereof. Thus, embodiments of the invention employ perspective and intuitive analysis protocols which do not convert all accelerations and angular velocities into a global coordinate (GPS) system and can correlate with lower computational load and more efficient analysis.

The terms “waveform activity signature(s),” “waveform signature” and “activity signature” are used interchangeably and refer to a waveform or waveform extracts of data output from one or more sensor channels of a multi-channel IMU while an individual is performing a specific or defined activity, typically associated with an ADL. Waveform signatures for a physical activity or action typically include activity signatures in both earth-based coordinate system waveforms (e.g., vertical acceleration) and in waveforms of the body-axis system.

The terms “waveform template” and “template” are used interchangeably and refer to a reference or defined waveform profile with one or more waveform activity signatures that are associated with a particular defined action, activity or ADL that can be used to identify or help identify a particular action or activity of a user. A waveform template may be provided based on a set or collection of activity waveform signatures of a particular action or activity obtained from a population norm or a (selected) plurality of individuals performing a defined (the same) action, activity or ADL. The waveform template can combine the collection, set or (sub) population of different waveform signatures of a respective activity, action of ADL in any suitable manner such as mean or average. A waveform template can be based on a baseline waveform profile or baseline composite profiles of a respective user taken at one or more times. A single waveform signature or an average or mean of waveform signatures obtained over time of a particular user can also be used to form a waveform template. For example, a single waveform signature of a user can form part or all of a waveform template when a user's IMU data is being compared with his/her own prior data of a specific or defined action/activity in a longitudinal comparison study fashion. Customized waveform templates may be used by combining reference signatures from selected groups, such as those having certain characteristics in common with a user, such as gender or age, as will be discussed below.

The terms “without gravity” or “exclude gravity” with respect to waveform profiles of body-coordinate systems means that gravity is mathematically factored from overall accelerations in order to get the accelerations and angular velocities of the torso caused by only overall body movements or physical interactions with the local environment (e.g., heel-strike or toe-off during gait cycles). As noted above, IMU instantaneous orientation quaternion data can be used to factor out the acceleration due to gravity. This can reduce computational load. It should be noted that the ability to factor out gravitational acceleration on a continuous instantaneous basis is not possible when using inertial sensor (IMU) packages which do not provide orientation data as part of their continuous data output (e.g., packages composed only of accelerometers and/or gyroscopes). Elimination of gravitational acceleration is typically carried out as a first step as a prelude to inertial navigation (i.e., dead-reckoning) computations and this step is included in the embodiments of the current invention. The instant invention does not require (and typically do not use) navigation equations and magnetometer or related global positioning sensor output data for activity monitoring, detection or performance evaluation since it is not necessary to track the position of an individual in 3-dimensional space in order to determine what physical activity is occurring or has occurred. In some particular embodiments of the current invention, however, global positioning calculations may be used to determine a location of a user as a secondary optional function in addition to activity monitoring.

Examples of commercially available IMUs of the type which can be used in embodiments of the invention include, for example, the Opal™, Emerald™ and Sapphire™ miniature sensor packages from APDM, Inc., Portland, Oreg. These devices are MEMS IMUs with varying wireless capabilities that provide streaming of data at a limited range to a computer-connected “access point” for up to 8 hours in the case of the Opal™ for real-time kinematic analyses or that provide onboard data storage during remote data collection for later download to a computer-connected “docking station” in the case of the Sapphire™ for the purpose of unlimited-range free space kinematic monitoring.

Another example of a suitable IMU includes the 3DM-GX3® 25™ from Microstrain Sensing Systems, Williston, Vt. The 3DM-GX3® 25™ is a high-performance, miniature Attitude Heading Reference System (AHRS), utilizing MEMS sensor technology. It combines a triaxial accelerometer, triaxial gyroscope, triaxial magnetometer, temperature sensor, and an on-board processor running a sophisticated sensor fusion algorithm to provide static and dynamic orientation, and inertial measurements.

Other MEMS technology IMUs may also be used, an example of which is the VN-200 from VectorNav Technologies, LLC, Dallas, Tex. This IMU incorporates a wide assortment of inertial sensors including a 3-axis accelerometer, 3-axis gyroscope, 3-axis magnetometer, and a barometric pressure sensor along with GPS capability. In some embodiments, where IMUs have the capability, one or more of the IMU's sensor features (i.e. barometric pressure sensor or GPS tracking) may not be used, may be inactivated, blocked or the data not evaluated. In other embodiments, the additional input maybe used for supplemental evaluation of a user, secondary to activity monitoring or identification, (e.g. studies of the influence of weather or other environmental conditions on physical motion/activity).

It is also contemplated that a smartphone, electronic notebook or electronic notepad such as an APPLE iPhone® smartphone, ANDROID® smartphone or other smartphone or other pervasive computing device platform can be configured with an onboard (integrated) IMU or can be configured to cooperate with a subject-worn minimal capability IMU to provide the motion data storage and/or part or all of the activity detection, identification and performance evaluation analyses of the motion sensor data. Output of the analysis of the motion detection and identification can also be transmitted to a smartphone, electronic notebook or the like as will be discussed further below.

The term “free space” refers to environments that are not required to be a static predefined space with a fixed static coordinate system and the free space can change or extend over more than one room to allow users to be monitored in natural areas in different environments without requiring a dedicated monitoring room and that does not require cameras, audio sensors, vibration sensors or similar external sensors in the environment for the motion detection associated with detection or identification of a physical activity or action.

The phrase “activities of daily living” herein abbreviated as ADLs refers to physical activities including, but not limited to those involving gross body mobility and stability functions such as are evaluated using batteries of tests such as the Berg Balance Scale or the Tinetti Performance Oriented Mobility Assessment. These daily functions include, but are not limited to gross physical activities such as “walking,” “standing turns and turns while walking,” “sit to stand” and “stand to sit” function/transfers, “pick up object,” “functional reach,” “step ups,” “lying down,” “stair climbing and descent.”

The term “primary signature” refers to a brief extract of one of the three acceleration or the three angular velocity IMU output data stream waveforms versus time (body-coordinate system) which is obtained during the performance of a particular physical activity and/or action. While ADLs can be composed of a series of individual body segment movements performed in concert, the “primary signature” is indicative of the body-fixed system acceleration or angular velocity activity waveform extract that represents the most consistently recognizable activity signature of the six waveforms when concurrently assessing multiple users and/or the waveform extract that statistically demonstrates the largest excursions from waveform baseline during performance of the target activity by multiple users at similar speeds. The duration of these primary signatures can also be indicative of the time required to perform a specific activity. For example, the functions sit-to-stand, stand-to-sit and picking up an object from the floor all typically involve significant torso flexion/extension in performing the activity. Therefore the body system y-axis angular velocity data extract during these activities would be considered the primary signature for these activities. Primary signatures for an activity can also be combined (e.g., averaged or mean) over multiple users or a specific subject population resulting in a waveform “primary waveform template” for a particular population and action or activity.

Embodiments of the invention can employ a hierarchy approach to identifying a physical action or activity using a primary waveform template with a secondary waveform template and optionally a tertiary and/or further serially ranked waveform template, allowing for a larger error threshold in the primary waveform template to identify an activity or a class of activities with similar primary templates. For example, concurrent data extracts from vertical acceleration waveform profiles can be used to help identify which of the three flexion/extension-related activities is actually being or was performed by means of a Boolean template matching technique; the vertical acceleration extract(s) are then considered as “secondary signatures or templates”. If a “pick up object” activity is identified, a concurrent clockwise or counterclockwise angular velocity extract about the body-fixed x-axis can identify whether the right hand or left hand was used in the activity. This waveform extract would then be considered a “tertiary signature” with regard to the “pick up object” activity during Boolean identification, since two other signatures would have to be matched or “true” before the third is considered. Note, however, that in the body-fixed coordinate system all of the accelerations and angular velocities are independent of each other which is extremely important in simplifying the data analysis algorithms and reducing computational load. Embodiments of the invention can thus be configured in this fashion so that the system/circuit/processor can identify, from monitored acceleration and angular velocity waveform signatures and predefined templates, what activities are being or have been performed by a user much like a cardiologist assesses heart electrical patterns and function by looking at multi-lead ECG tracings.

The term “patient” or “subject” refers to a user being monitored or examined. The user is typically human. However, it is contemplated that embodiments may also be configured for animal monitoring.

As used herein, the terms “substantially real time” and “near real time” are used interchangeably to mean within about 5 minutes, typically within about 0.1 second to about 2 minutes of when a user actually performs a physical activity.

The term “automatic” means that substantially all or all of the operations so described can be carried out without the assistance and/or manual input of a human operator. The term “electronic” means that the system, operation or device can communicate using any suitable electronic media and typically employs programmatically controlling the communication between participants using a computer network. The term “programmatically” means the action is directed via computer program code. The term “hub” means a node and/or control site (or sites) that controls and/or hosts data exchange between different user sites using a computer network. The term “HIPAA” refers to the United States laws defined by the Health Insurance Portability and Accountability Act.

The terms “healthcare data” and “clinical data” and “patient records” are used interchangeably and include any and/or all of treatment, medicinal, drug or prescription use, laboratory tests and/or results, diagnostic information, personal information, insurance information other relevant data associated with a patient.

The term “APP” refers to a computer program configured to provide defined functionality on a computer including pervasive computing devices such as an electronic notebook or notepad, smart phone, lap top, and the like, typically accessible via an icon on a display.

The terms “web-based”, “online” or “cloud-based” mean that the service is available using the World Wide Web (Internet), typically via at least one server to communicate with different users. The communication protocol can include hypertext transfer protocol (HTTP).

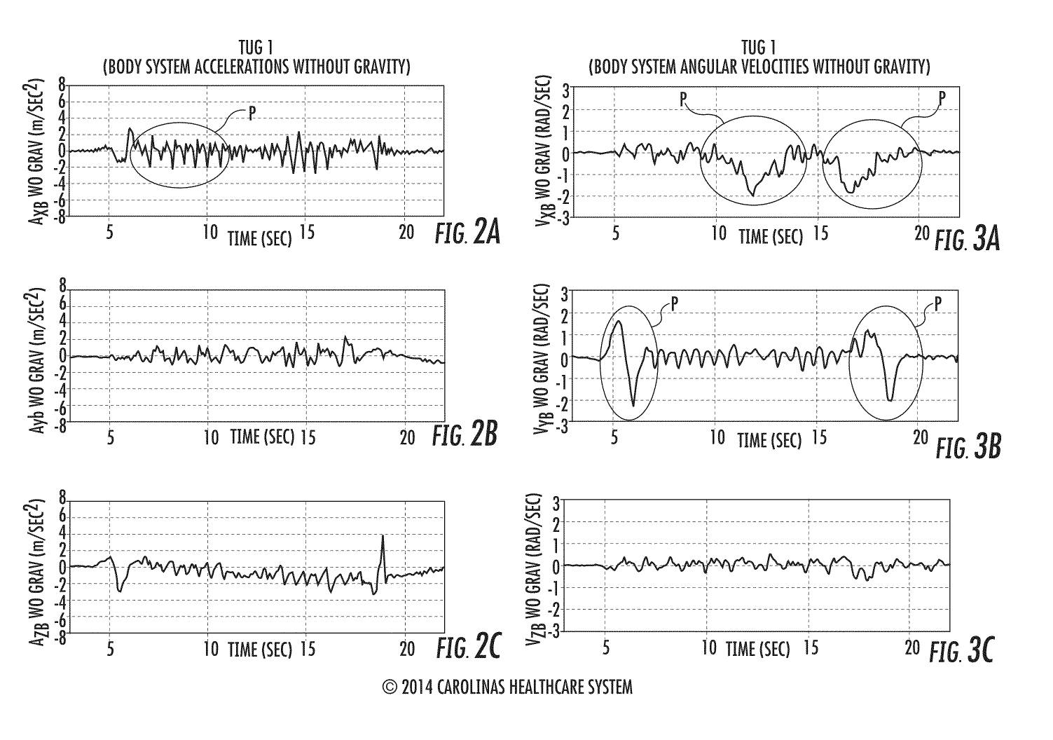

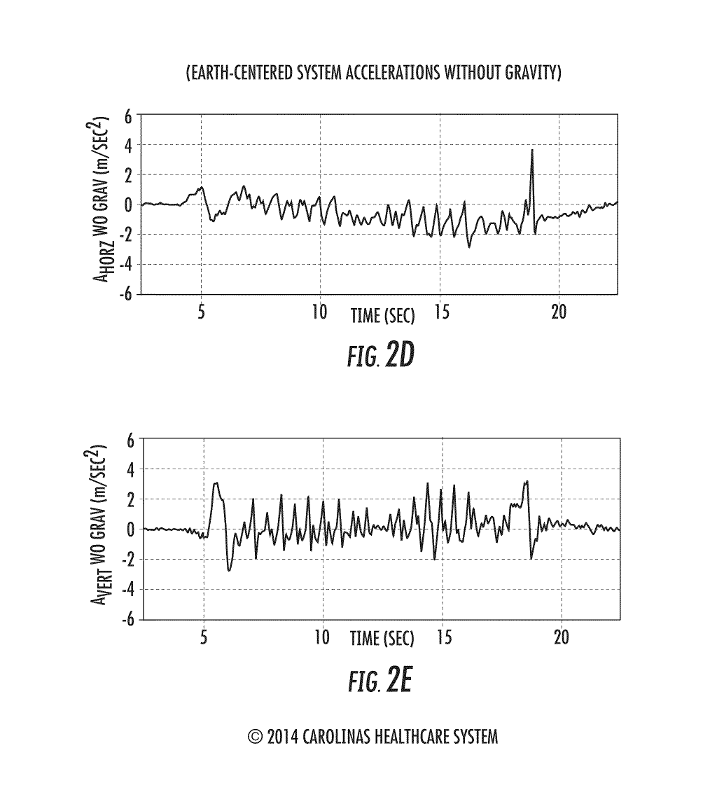

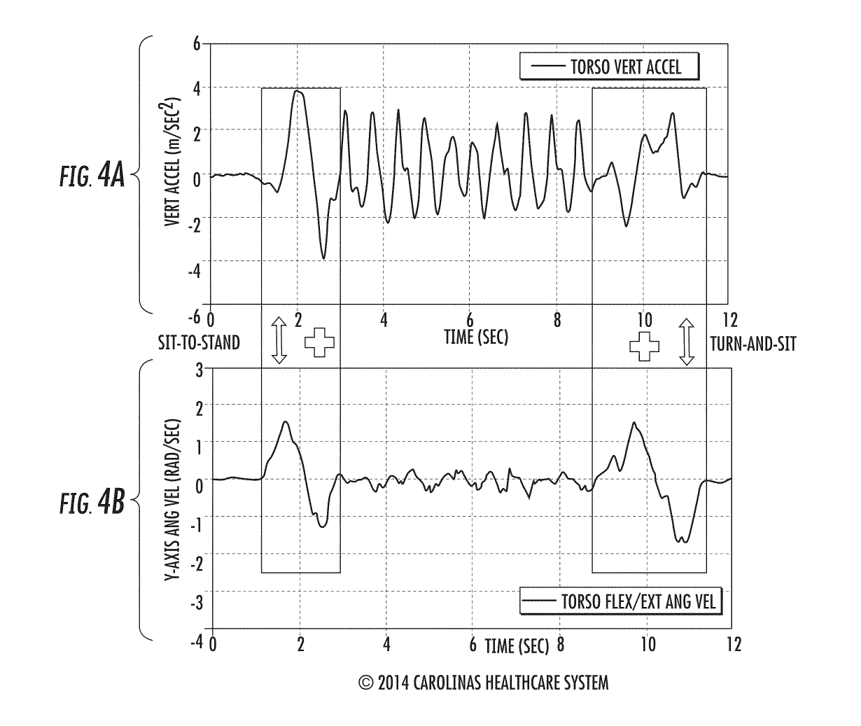

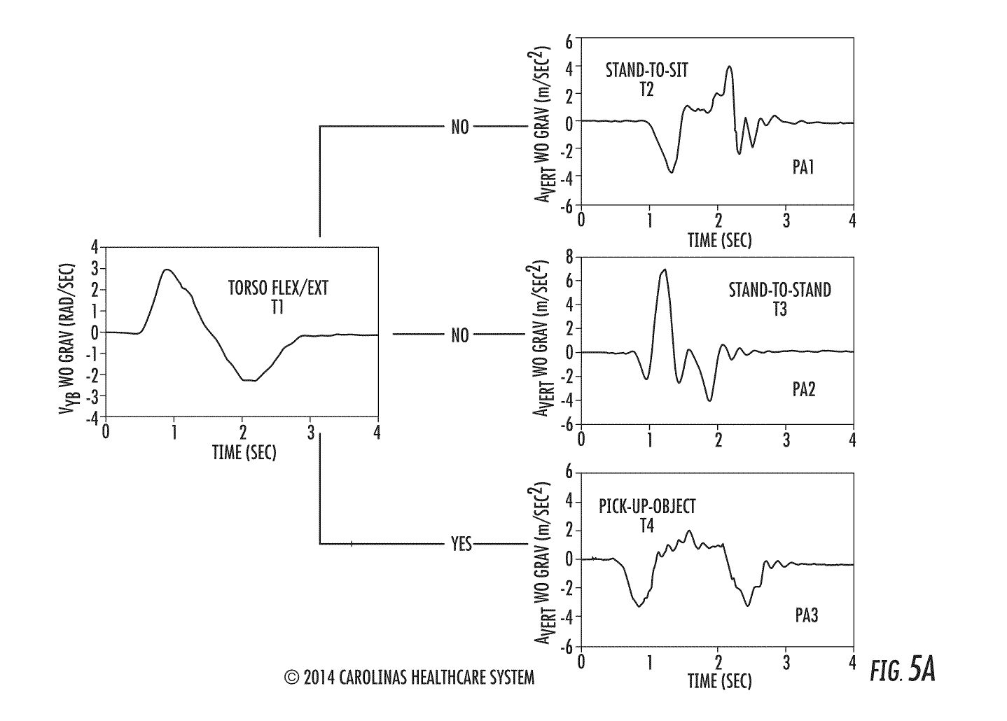

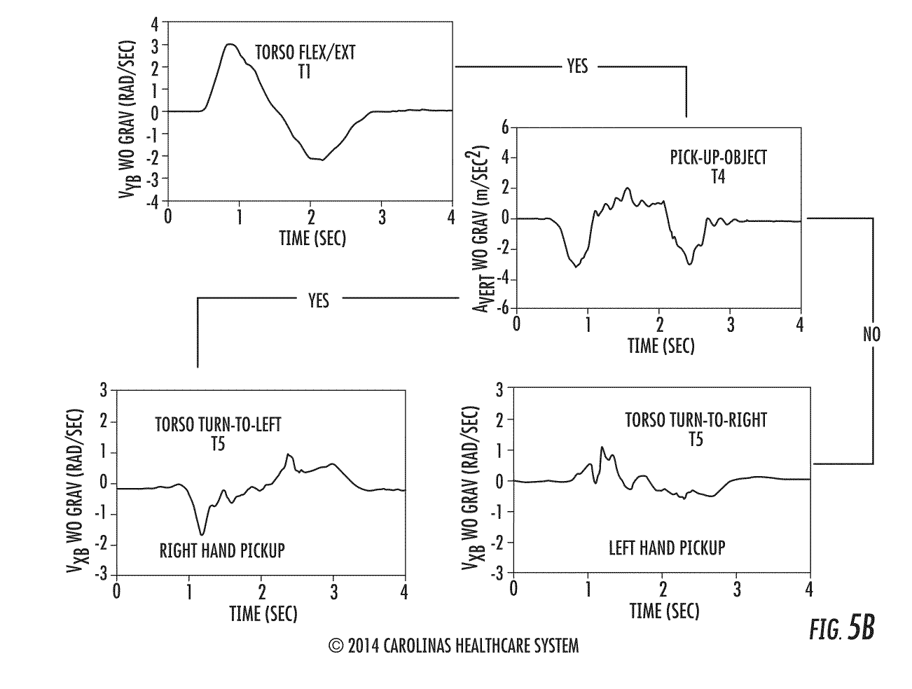

Briefly stated, embodiments of the invention use digital data from a plurality of different active sensor channels from an IMU for activity detection, identification and, optionally, performance evaluation, in order to reduce computational and/or data storage requirements as compared with other methods of performing these functions. The IMU data can be used to generate at least six different graphs/waveform profiles directly from the accelerometers and gyroscopes (body axis system), three acceleration graphs (e.g., FIGS. 2A-2C) and three angular velocity graphs (e.g., FIGS. 3A-3C). Additional orientation quaternion data can be used to also generate earth-centered vertical and horizontal acceleration data (e.g., FIGS. 2D-2E).

In the past, Zhang et al. proposed the use of three orthogonal accelerometers (i.e. a sensor node) on each moving body segment and a template matching approach to perform analysis of upper extremity rehabilitation activity. In this approach, Zhang et al. follow the prior teaching that for activity monitoring, as viewed as a type of motion tracking, each moving body segment needs to be instrumented. This approach has a number of significant drawbacks. Zhang et al. acknowledges that as the accelerometer sensors move in 3-space, the orientation of the sensors versus the gravitation direction changes and states that this is a useful feature. In fact, when using only accelerometers without sensor package orientation data, it is impossible to factor out gravitational acceleration even if desired. For many activity monitoring cases, the magnitude of gravitational acceleration is large compared to the moving body segment accelerations. Hence the signal (activity accelerations) to noise (gravitational accelerations) ratio is very low. Thus, Zhang et al. does not teach or suggest the use of current IMU technology which utilizes sensor fusion techniques and orientation data generation and teaches away from identifying physical activity/action using motion data that excludes gravity. See, e.g., Upper limb motion capturing and classification for unsupervised stroke rehabilitation, IECON 2011-37^{th} Annual Conference on IEEE Industrial Electronics Society, pp. 3832-3836, 2011; and Template matching based motion classification for unsupervised post-stroke rehabilitation, Bioelectronics and Bioinformatics (SBB) 2011, pp. 199-202. Also, in this approach, each triaxial accelerometer node on each moving body segment produces and requires three concurrent acceleration templates to identify a specific movement for that moving body segment. Thus, for any specific arm motion (i.e. upper arm and forearm), Zhang et al. use six concurrent templates (i.e. from two separate triaxial accelerometer packages placed on two body segments) to identify that motion. Furthermore, with this approach, accelerations of the proximal arm segment affect the accelerations of the distal arm segment. Small variations in proximal segment accelerations in addition to rotations at joints between the two sensor nodes can produce much larger acceleration variations in the dependent distal segment acceleration waveforms due to the moment arm between the two sensor nodes. This dependency increases the computational load with any variability in the accelerations due to motion. Finally, the use of acceleration templates alone, particularly with intra-segment dependencies do not support an intuitive interpretation of the data, such as is possible with one moving segment and independent body-fixed acceleration and angular velocity data according to embodiments of the invention. Intuitive data assessments allow for the development of enhanced algorithms such as Boolean template matching techniques in order interpret variations in methods by which a particular activity can be performed.

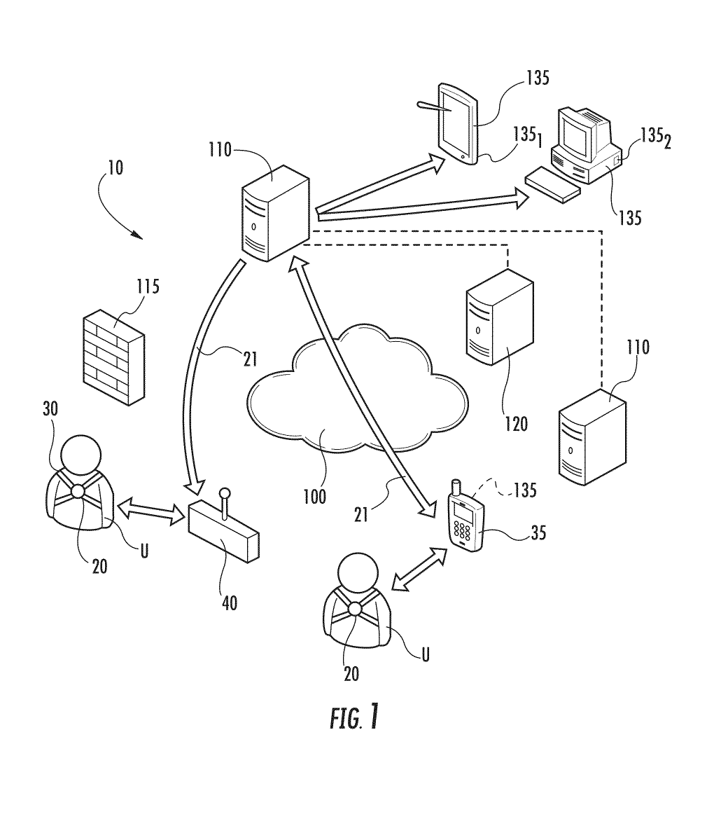

Turning now to the figures, FIG. 1 illustrates an exemplary monitoring system 10. As shown, the system 10 can be a multi-user system that can concurrently collect motion data from a plurality of different users U. Each user U will have an IMU 20 placed above the waist (above the user's center of gravity), preferably on a front surface of the upper torso, proximate or over the sternum. The system 10 can be configured so that each user U is required to have only a single IMU placed proximate the sternum, thus promoting ease of wearability and compliance in use, particularly in free space for ADL continuous monitoring. However, more than one IMU may be used. If so, the IMU data from each IMU can be evaluated separately or may be averaged or otherwise combined to generate the user waveform profiles.

In some particular embodiments, the system 10 can use a single IMU 20 for a respective user U that can be placed, typically substantially laterally centered, over an upper portion of the sternum body, more typically over the manubrium. The at least one (or single) IMU 20 can be placed above the waist (above the user center of mass) to have more sensitivity to rotation relative to waist mounted IMUs providing a better torque arm placement/position. It is contemplated that placement of the IMU 20 over a top portion of the sternum can position the IMU in a body segment with the center of mass where there is minimal soft tissue and where certain motion/action such as ground reaction forces can be transmitted through the skeletal system to allow a single IMU to have sufficient sensitivity for detection of the different motions of body parts. As noted above, more than one IMU may be used. If so, the multiple IMUs can be on the same body segment (e.g., all on the torso) or on different segments depending on the specificity of activities to be monitored.

As shown in FIG. 1, motion data 21 from the IMU 20 can be transmitted to a remote site via the internet 100. Typically, digital motion 21 from the IMU 20 is transmitted to at least one web server 110. Firewalls 115 may be used for privacy, particularly where the motion evaluation is for medical purposes. Evaluation of the motion data can be carried out using at least one server 110. Monitoring results, alerts or other related information can be transmitted back to a respective user U and/or to one or more other defined location or person, e.g., a monitoring service, clinical facility, doctor, nurse, or other clinician, caretaker or other authorized person. As will be discussed further below, the evaluated data can be sent to any device including a land-based (POTS) telephone, electronic tablet, electronic notebook, computer, or smartphone. The data 21 can be wirelessly transmitted or transmitted via the Internet to the server. However, the motion data 21 can also be collected using data storage capabilities onboard the IMU, physical storage media such as memory sticks or cards or data storage onboard another device connected to the IMU, with the data subsequently downloaded or reviewed.

Embodiments of the present invention can electronically automatically apply waveform template matching analysis protocols to acceleration and angular velocity data which are obtained from motion and orientation sensors within the IMU 20. FIGS. 2A-2C are examples of concurrent waveform profiles of acceleration (m/sec²) for x, y, z body system accelerations without gravity for a series of events/activities performed as part of an EATUG test. FIGS. 3A-3C are examples of concurrent waveform profiles of body system angular velocities without gravity (rad/sec) for the same series of events/activities (t=0 to t=20 seconds). Certain of these waveform profiles have a primary signature P associated with a particular physical activity (marked with the circles labeled P). Note that for a specific activity only one primary signature is identified on (i.e. is germane to) one specific data stream. In FIG. 2A, the signature for “walking or gait” is identified on the body system x-axis acceleration graph. Two signatures for “walking turns” are identified on the body system x-axis angular velocity graph in FIG. 3A. In FIG. 3B, primary signatures are identified for “sit-to-stand” and “turn-and-sit” activities on the body system y-axis angular velocity graph. Thus a specific template evaluated v. a specific data stream is used to identify a specific activity and similar signatures identified on different data streams are indicative of different activities. Thus, in some embodiments, only specific primary templates P are evaluated v. specific sensor data streams to identify the physical action/activity.

The system 10 includes an activity signature database(s) 120 (FIG. 1). To generate templates with activity signatures, data can be collected from motion sensors and orientation sensors, such as multi-directional accelerometers, gyroscopes and magnetometers which are typically contained in a single IMU 20 attached to an individual while the individual performs specific preselected physical activities. The sensors' activity waveform signatures produced in this way can be overlaid with activity waveform signature data from other individuals performing the same activity. The waveform templates can be generated using multiple individual's activity signatures for a particular activity or action and/or by using one or more prior performance monitoring of a specific user during a particular activity or action. The waveform templates may be average or mean waveform templates of a series of concurrent waveforms from all of the sensors' data streams of a reference population that can be constructed to serve as templates for specific actions and/or activities. Activity waveform templates for different activities and/or activity waveform templates for the same activity using different subject populations can be constructed and stored in the template database in an electronic template library.

Activity template(s) and any continuous extended-duration raw data file representing random activities are digital data files comprise of various sensor data streams v. time at given time intervals. In some embodiments, the system can employ an analysis process to detect specific events that occur in near real time during sensor data acquisition or have occurred during data acquisition and are slated for detection, identification and evaluation at a later time. By way of example, the digitized sensor data v. time extracts of different activity templates, comprised of “n” sensor data points, can be compared in iteratively increasing time steps v. continuously running or stored sensor data streams and with each step, the template(s) data can be subtracted from the raw data and a mean square error can be calculated. Equation (3) is a representative equation used for mean square error data calculations for two waveforms. In this case, one of the waveforms is the activity template and the other is an iterative extract of the (continuously or periodic) running or recorded data stream for a sensor channel with an equal number of equally-spaced data points (N), where: (Xs,Ys) is the sensor streamed data and (Xt,Yt) is the template data, MSE is the mean squared error between the template and the iterative extract of streamed data.

$$MSE = \frac{1}{N} {\sum }_{i = 1}^{N} {( {Ys}_{i} - {Yt}_{i} )}^{2} \qquad Equation 3$$

When the mean square error falls below a predetermined threshold, the template and iterative extract raw data are determined to be a match and an activity is detected and possibly identified. Further localized sequential centering iterations can be used to result in a minimal mean square error between the template and raw data and this value can serve as an indicator of the goodness-of-fit of the template and raw data (zero error would indicate an exact fit). Equation 3 represents one example of an error minimization algorithm that can be used for event detection, identification and evaluation. Numerous mathematical algorithms exist, however, that can be used for template matching based on waveform difference minimization or maximization of waveform interactions.

Any number of activity or action waveform templates can be evaluated against digital (raw) data from channels of an IMU attached to respective individual users U in any medical setting and/or in the community. The system can provide quantitative information with respect to different activities that are performed and how well they are performed (i.e. based on the template(s) selected for comparison purposes).

Waveform data can be analyzed to periodically or continuously update the database of templates for subsequent analyses. The waveform data can be raw, filtered or partially filtered. This programmed “learning” can allow waveform(s) template(s) for individual activities, which can be collected based on defined individual characteristics (age, gender, etc) for electronic selection and/or sorting, to become more refined and can also allow template matching error limits to be adjusted so as to be more indicative of standard deviations and/or additional standard deviation waveforms can be established in order to assess normal variations in activity performance in different populations.

In some embodiments, the system 10 can be used to determine not only when an activity is performed but also what specific activity is being performed and how well the activity is being performed. This activity analysis and assessment can be based on identification of specific waveform patterns correlated to specific physical actions or activities from the accelerometer, gyroscope and orientation data obtained from the motion sensors of a respective IMU. Physical motion or activity waveform templates, typically stored in one or more electronic memories and/or databases, can be used for specific event detection when electronically reviewing (long-running) data files from the IMUs. The electronic review can be carried out post-activity or in near real time.

In some embodiments, the system can be configured to identify when a specific activity is detected and the degree to which an individual/user U waveform profile “fits” with a defined correlated activity waveform template(s). The system 10 can electronically automatically score or otherwise evaluate (e.g., “good”, “poor” and the like) the user's motion based on the comparison to reflect how well (how “normally”) the activity was performed. The system 10 may be configured to employ a baseline template(s) based on activity signature data from the actual user during subsequent physical activity to assess variation from that baseline(s) templates. This information can be used, for example, to indicate a need for medical evaluation for potential physical therapy or a change in medication and the like.

The system 10 can be configured to electronically score one or a plurality of physical actions using conventional or standardized fall potential and/or balance tests. For example, the system can be configured to generate an EA (electronically augmented) Tinetti Balance Tests (TBT), using predefined waveform templates. In some cases, template matching can automate a scoring process of standardized balance or fall potential tests and remove observer subjectivity, quantify and/or provide a continuous scoring scale (i.e., continuous v. interval measurement scale) or hasten the process when scoring known test activities such as the individual activities in a Tinetti or Berg series of tests or combined activity tests like the TUG or repetitious test activities like the Five Times Sit-To-Stand test. In these instances, it is known when the activity is performed and what activity is performed; thus, the systems/methods can be configured to assess how well it was performed. In these circumstances, the error generated in performing a template match for the activity v. some “norm” template can be used to rate or score the activity or multiple template matches and multiple errors can be combined in various ways to get a more complete analysis of how well an activity was performed v. the “norm”.

The scores can comprise a numerical composite score of between 0-10, along with sub-scores for defined individual physical actions. The sub-scores can be standardized to correlate with a current numeric scoring system, e.g., between 0-2 for each evaluated action, like the conventional subjective “manual” scoring of a Tinetti Balance test. The system 10 may also be configured to generate a composite score for physical motion actions of Tinetti Gait Tests (TGT) and generate sub-scores for each of seven actions: (a) initiation of gait (0-1): (b) step length and height—right swing length, right swing height, left swing length, left swing height (each 0 and 1); (c) step symmetry (0-1); (d) step continuity (0-1); (e) path (0, 1, 2); (f) trunk scored based on three characterizations: with no sway/flexion/arms; walking aid or no sway but flexion knees/back; arms or marked sway, walking aid (0, 1, 2); and (g) walking stance (0, 1). The gait test can be carried out at normal and/or max possible speed.

The system 10 can be configured to electronically score a functional reach test at three trial distances to assess when a patient's feet lifted up from the floor or he/she fell forward. If the reach is less than 6 or 7 inches, this indicates limited functional balance.

The system 10 can include, but is not limited to waveform profile templates for Berg Balance Tests (BBT), typically scored between 0-4, for activities not covered by the TBT or TGT such as, (a) ease/balance in picking up an object and (b) placing alternate foot on a step or stool while unsupported (8 step repetitions).

The system 10 can electronically generate an overall assessment of low, moderate or high falls risk using one or more of the automated scores. The standardized TBT, TGT and BBT tests (i.e. activities such as sit-to-stand, stand-to sit, standing and walking turns, gait, retrieve an object from the floor, functional reach, etc) are routinely used to evaluate an individual's ability to perform ADLs. Thus, embodiments of the invention can electronically automatically quantitatively analyze motion sensor data to evaluate standardized tests in a clinical setting or analyze ADLs in a community setting providing for a less subjective evaluation.

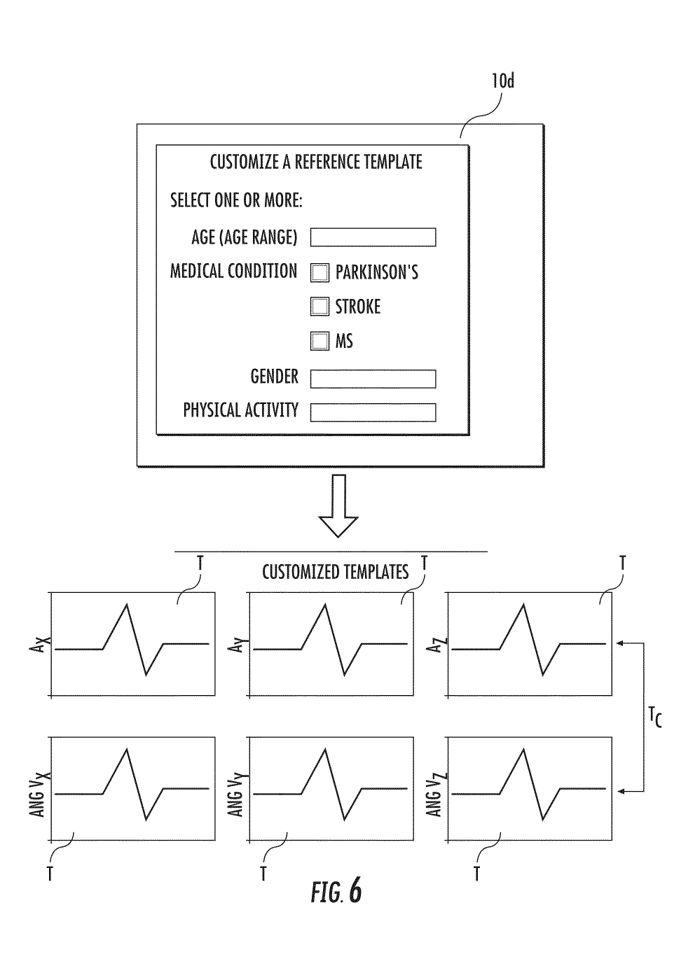

Note that some actions or physical activities have patterns in the waveform profile that can be similar. FIGS. 4A and 4B show two concurrent pairs of activity templates useful for detection and identification of sit-to-stand (left) and stand-to-sit (right) activities performed during an EATUG test. Thus, for example, the beginning (sit-to-stand) and end (turn-and-sit) patterns look very similar (FIG. 4B) with regard to flexion/extension angular velocity patterns of the torso about the body system y-axis (side to side axis). However, the concurrent vertical acceleration patterns (earth-based coordinate system) waveform templates (FIG. 4A) are very different for the sit-to-stand and the turn-and-sit activities. Hence while a single flexion/extension template may detect an event and identify it as possibly either a sit-to-stand activity or a turn-and-sit activity, vertical acceleration templates can be used as secondary templates to delineate between the two possible torso flexion/extension activities. This represents a basic Boolean template matching technique where two templates are used to identify an activity from a set of possible activities.

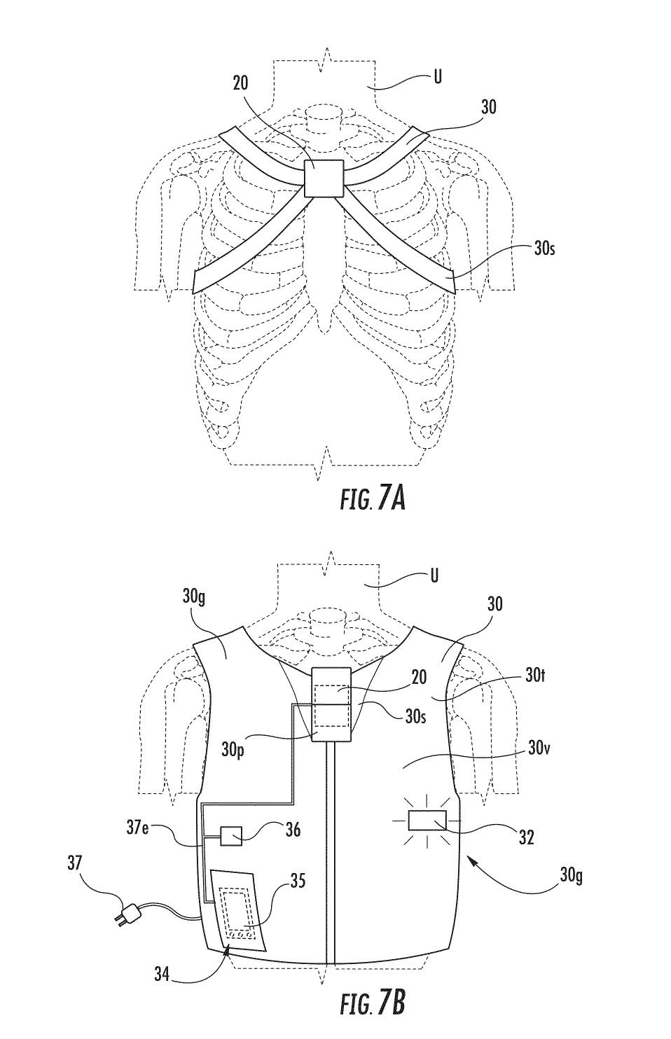

As schematically illustrated in FIGS. 5A and 5B, embodiments of the present application can be configured to employ a template matching approach which typically utilizes more than two waveform profile templates. In FIG. 5A, a body system y-axis torso flexion/extension template T1 and a vertical acceleration template T4 are used to identify the correct activity as bending at the waist to pick-up-object (PA3) from a subset of three possible torso flexion/extension-based activities.

In FIG. 5B, primary template T1 is used with secondary template T4 and tertiary templates T5 and T6 to identify one of two possible variations of that activity where the object is picked up using the right hand (T5) v. the left hand (T6) using a Boolean template matching technique. A Boolean activity evaluation can employ between 1-8 templates of associated acceleration and angular velocity waveform templates for any action/physical activity having one or more activity signatures (e.g., event). Once an activity is properly identified, template matching errors can be used to evaluate “goodness-of-fit”. A goodness of fit evaluation can also or alternatively be carried out for each template match before a PA (physical activity) is actually affirmatively identified.

The Boolean technique permits large allowable error thresholds to be used in initial template matching in order to detect a subset of potential activities while multiple concurrent templates can be used to positively identify the specific activity being performed and determine any particular variation that were used in performing that event.

Most activities of daily living involve relatively few primary motion patterns which combine to form more complex functions. A relatively simple timed up and go (TUG) test involves four primary motion activities (i.e. sit-to-stand, walking, turning and turn-and-sit). The system 10 can be in communication with or have an electronic library or database with the associated waveform (reference) templates for the any defined series of activities associated with a motion or stability assessment test. Electronic template libraries or databases can also be arranged in a defined hierarchy so that secondary, tertiary, etc. templates are arranged in cascading fashion stemming from primary templates to facilitate the efficient identification and/or evaluation of performed activities.

The system 10 can have an electronic waveform template library constructed with as few or as many activity templates as appropriate for a targeted activity monitoring task.

For activity monitoring, the systems/methods/programs can be configured to detect specific digital activity on a running waveform at near-real time or on a running waveform during playback. In both cases, activity monitoring potentially involves detection of an activity (easy), identification of that activity (more involved) and, where used, performance evaluation for that activity (still more involved). The activity analysis depends on the application and the analysis complexity required for that application. However, template matching and using matching errors can be provided to work across many applications.

In some cases, such as walking, one template (e.g. using vertical acceleration or body system x-axis (up-down) acceleration data) can be used for detection, identification and a rough performance evaluation (where desired). However, more templates may be required for better performance evaluations such as templates that indicate side-to-side sway (i.e. y-axis acceleration templates or z-axis angular velocity templates). This isn't Boolean template matching but simply the use of more templates to evaluate performance.

In other cases, there are templates that are prominent (primary) for a class of activities, such as activities that often performed with significant torso flexion/extension (e.g. sit-to-stand, stand-to-sit, pick-up-object). In this case one flexion/extension template (i.e. angular rotation about the body system y-axis) might “detect” all three activities if the allowable error is high in template matching. Here a second (secondary) template (e.g. vertical acceleration) can be used to identify the specific activity, say, pick-up-object (i.e. template matching error is smallest for that activity). There are variations as to how the activity can be performed (i.e. right hand used or left hand used or left hand). These can be delineated using body system x-axis angular velocity templates. Thus, Boolean template matching is when multiple templates are used to identify exactly what activity was performed and/or how (i.e., using what variation of the activity) was it performed. A performance evaluation can involve analyzing both “how” it was carried out and/or “how well” an activity was performed. “How well” can be evaluated as the error between the (reference) template(s) and the detected waveform(s) extracts when performing template matching.

Another example of how this approach can be applied relates to sit-to-stand or stand-to-sit activities either with or without using your hands and/or using one hand or both hands. The Boolean approach can have a hierarchy or predetermined order in which the templates are applied to corresponding user waveforms determine specific activities and variation in those activities (i.e. use of primary, secondary, tertiary, etc. templates).

In general, in certain activities, one or two template matches (Boolean matching) are all that is needed for activity detection and identification and one or two template matches can provide a rough (or first order) evaluation of performance based on template matching errors. However, a more complex analysis of variations in how an activity is performed and how well it is performed generally requires three or more concurrent template matches (i.e. more Boolean match steps and calculations of template matching errors).

For activity monitoring, there are always a number of templates being continuously scanned during a physical event, e.g., the five acceleration and three angular velocity data channels in search of activities. For chronic monitoring, the scanning can be carried out in near real time but may be discontinuous rather than continuous (e.g., between a continuous monitoring mode and a ‘sleep’ mode). In most cases, a particular template only applies (i.e. identifies an activity or an activity variation) to a specific data channel. Hence not all templates have to be continuously scanned for all data channels. Some template shapes, however, do apply to multiple data channels and can be continuously scanned on multiple channels during certain time frames or continuously when “on”.

In some embodiments, mean squared error assessments of template matching to user waveforms can be used as shown in Equation 3 below. However, other statistical mathematical evaluations can be used to determine a “match” or a “mismatch” by minimizing or maximizing the mathematical difference between the template and scanned waveform through some form of addition, subtraction, multiplication or division or related higher power functions with some sort of error threshold that can be used to define a match or mismatch. Error thresholds may be different for different templates or activities and error thresholds can depend on the signal variability in performing the activity.

Where used, Boolean template matching can allow initial allowable error thresholds or similar distinguishing numerical thresholds to be can be set to detect variable activities and multiple templates can be used for positive or affirmative activity identification and performance evaluation. This configuration can be such that fewer activities are missed and/or incorrectly identified.

In some embodiments, the system 10 can include at least one database 120 (FIG. 1) with motion data representative of specific activities performed by different individuals (for human use) and different animals of like size for animal use. The templates can be stored as prepared templates or stored in waveform signature files that can be accessed to generate the waveform templates T. For human use, the motion data for the activity signature waveforms or waveform templates can be obtained for a specific activity from individuals of different ages, genders, and/or different medical conditions.

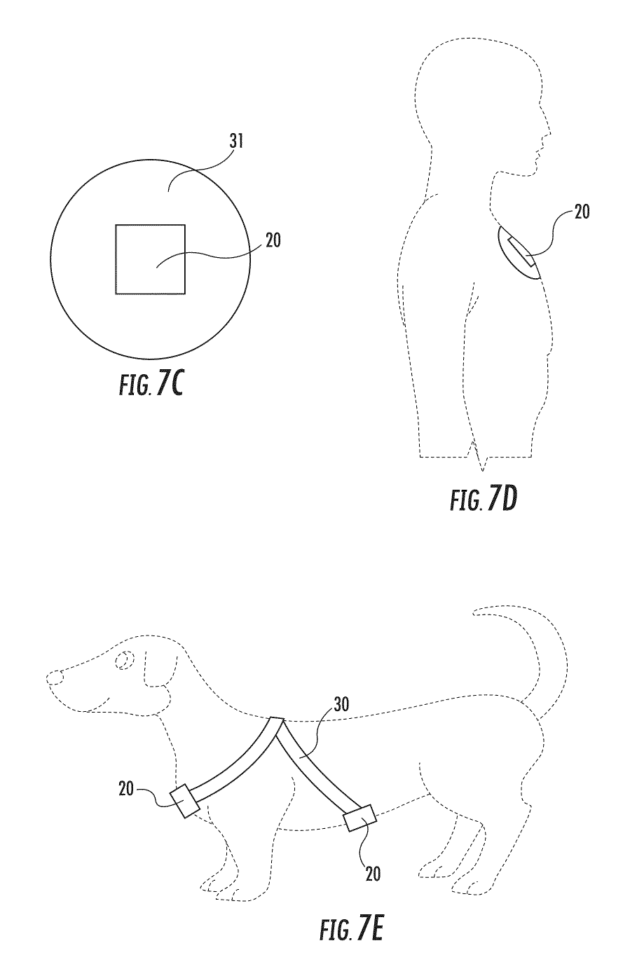

FIG. 6 illustrates that the system 10 can be configured to allow activity templates to be customized or created “on-the-fly” for any defined groups or populations of individuals. The term “on-the-fly” refers to an electronic automatic selection of relevant waveform templates of a sub-group of available waveform templates based on defined or selected parameters or criteria. This template generation process allows a more relevant template for a particular user customized for any age and/or medical condition to allow comparison of physical ability with an appropriate reference norm for individuals meeting the same selection criteria as the user. This customized or on-the-fly template construction approach can also allow for comparisons of activity performance for a given individual versus groups of individuals with any selection criteria deemed appropriate.

As shown in FIG. 6, the system 10 can provide a display screen or window 10d that accepts user input to select one or more defined categories, e.g., one or more of age (age range), medical condition or disease state, prescribed medication, gender and particular physical activity that can be used to generate the customized or on-the-fly templates Tc. The system can allow a clinician, researcher or other monitoring user to select a desired reference population or subpopulation to generate the on-the-fly or customized template for any number of activities by generating associated templates using activity waveform signatures only from those individuals meeting the selection criteria. The system 10 can also be configured to generate both “norm” activity templates (typically calculated as a mean or average of activity waveform signatures from a suitable control population) and/or customized templates from individuals similar to the user being monitored.

This approach also permits a basic assessment of how well the detected activity was performed by comparing the activity waveform(s) signature(s) to representative activity templates based on a variety of subject populations. These populations could include individuals of different ages, individuals with neuromuscular diseases, individuals recovering from multi-trauma conditions or individuals on specific medications as basic examples. In each of these cases, template matching can be used to compare how an individual's performance of particular activities compares to the performance of those same activities by similar or dissimilar subject populations.

The system 10 can be configured to electronically identify physical actions or activities such as a plurality or all of the following: step up (stair), step down (stair), walk, sit-to-stand, stand-to-sit, reach, pick up object, bend, stair climbing, step up (curb), step down (curb), stair descent.

Embodiments of the invention may be used to automatically monitor user transfer from one device to another such as from a bed to a chair, a chair to a toilet and the like allowing for reduced clinical support load in a nursing home, rehabilitation center or hospital, for example. The chair may include a wheelchair or personal motorized mobility device.

The system 10 can be used to monitor patients with activity restrictions in hospitals or nursing homes. The system 10 can be used in a clinic to assess a patient's progress post-injury or surgery or assess effects of pharmacologic or physical therapies. It is contemplated that the system 10 can be used for patient populations where kinesiologic assessment is particularly useful such as orthopedic trauma patients, patients with joint disease and joint replacements, as well as those with degenerative neuromuscular disease, traumatic brain injury or stroke. The system 10 can be used for activity monitoring to assess and/or titrate the dosage of drugs that have kinesiologic side effects. The system 10 may be used for remote patient monitoring to reduce unplanned hospitalization, ER visits, and improved patient self-care.

FIGS. 7A and 7B illustrate exemplary positions of an IMU 20 on a (human) user U using a wearable support 30. FIG. 7A illustrates that the support 30 can comprise a flexible chest strap system or harness 30s. The straps can be flexible and adjustable so as to accommodate different size users U and proper placement. FIG. 7B illustrates that the support 30 can be a garment 30g (shown as a vest 30v) that can have one or more pockets 30p that properly places the IMU 20 on the user U. The garment 30g can be configured to have at least an upper section 30t with increased snugness relative to other sections and/or configured with material having sufficient elastic stretch to allow the IMU 20 to be in snug contact with the user's skin, directly, or indirectly via a layer of thin material, so as to be able to detect motion of the torso and arms of the user U, typically using a single IMU. The garment 30g, such as vest 30v, can comprise a breathable conformable material that is elastically stretchable over at least an upper part of the chest region over the sternum to provide the desired snug contact with the user U.

The garment can optionally include sleeves and a sleeve can optionally hold another IMU (not shown). No leg IMUs are required which may promote user compliance for long term monitoring.

As shown in FIG. 7C, the IMU 20 may be an implantable device which may be a subcutaneuously implantable device residing proximate an outer surface of the skin or deeper, but over the sternum. As shown in FIG. 7D, the IMU 20 may be configured to adhesively attach to an outer surface of skin directly or with a patch 31 for example, rather than have it be positioned using a garment, strap or other support.

FIG. 7E illustrates that the IMU 20 and support 30 can be configured for use with an animal subject. The IMU 20 can include one positioned between the fore and rear legs on the upper abdominal region and/or in front of the forelegs, for example. The IMU 20 may be placed at other suitable locations so as to detect the motion for monitoring kinematics.

As shown by way of example in FIG. 7B, the IMU support 30 can include an optional audio, tactile and/or visual alert 32. The support 30 and/or IMU 20 can be configured to wirelessly transmit an alert to a remote site when no motion data is received for a number of hours or days. The support alert 32 and/or remote alert can be activated when there is no motion data from a user for a defined time frame, e.g., a number of hours or days, for example, to facilitate continuous compliance and/or identify a problem with the user U or system. The system 10 can be configured to generate a local and/or remote audible/visual alert when a fall condition may be increasing based on the motion analysis. The local alert may prompt the user U to take steps to prevent a fall. The alert can be in the form of a tactile vibration, sound (tone, alarm or voice) alert and/or flashing or visible signal.

The support 30 can include a receptacle 34 such as a pocket, strap or other receptacle for a smartphone 35. The support 30 can include a back-up power source 36 such as a battery, typically a rechargeable battery. The support 30 can include or releasably attach to a charger connection 37 and may include an onboard electrical path(s) 37e that can allow a user to plug in to a power source for recharging the IMU 20 without requiring removal from the support 30 and/or the back-up power source 36. The garment or support 30 adjacent the IMU 20 can include an electrical path that engages the IMU for charging or powering the IMU 20 over time or when needed.

The system 10 can be configured to accommodate users' monitoring of ADLs for substantially continuous run mode and/or for episodic monitoring during a physical therapy or medical evaluation exercise. The IMU 20 can be configured to be placed in a deactivated state and/or go into a sleep mode during periods of inactivity to promote battery life. The IMU 20 can be configured to fully reactivate upon an input or automatically upon detection of any movement.

FIGS. 7F, 7G and 7H illustrate the body-axis coordinate system B of the IMU as it is on the user U and FIG. 7H also shows the earth-based coordinate system E as discussed above.

Referring again to FIG. 1, a plurality of different users U at different residential, public or clinical sites can be in communication with the at least one server 110 hosting or providing the monitoring system 10. The at least one server 110 can allow access to multiple users U from different sites and/or the same clinical facility at any one time. The systems 10 can use a computer network with a distributed, client-server architecture. The system 10 can be accessed via any desired device having access to the Internet 100 including wireless communication systems (such as cellular telephones or smartphones), PDAs, desktop or portable computers including lap or handheld computers, electronic tablets or notebooks and the like. FIG. 1 illustrates, by way of example only, that a user U can wirelessly access the system 10 via a local access console 40 and another user U can wirelessly access the system 10 via a smartphone 35. The data stream is shown as two-way, but may also be in a single direction.

Embodiments of the invention may use a computing architecture in which the user interface, the application processing logic, and/or the underlying database(s) can be encapsulated in logically-separate processes. In any given application utilizing this type of computing architecture, the number of tiers may vary depending on the requirements of the particular application; thus, such applications are generally described as employing a n-tier architecture. See, e.g., Exforsys.com, N-Tier Client-Server Architecture. For instance, some embodiments of the invention may employ a 2-tier architecture, commonly referred to as a client-server architecture, wherein a client application such as a web browser makes a request from a web server, which processes the request and returns the desired response (in this case, web pages). Other embodiments of the invention may be structured as a 3-tier or other larger multi-tier architecture, wherein the web server provides the user interface by generating web pages requested by a web browser, which receives and displays code in a recognized language such as dynamic HTML (Hypertext Markup Language); middleware executing on an application server handles the business logic; and database servers manage data functions. Often, the business logic tier may be refined into further separate tiers to enhance manageability, scalability, and/or security.

Accordingly, in some web-based clinical pathway systems or hosted services, the web applications can use a 2-tier or 3-tier architecture with a presentation tier that provides the different clinical pathways. The web application tiers may be implemented on a single application server, or may be distributed over a plurality of application servers. The presentation tier can provide web pages that allow a user access using a common portal presented on local client devices such as local desk top or laptop computers, smartphones, electronic notebooks or personal computing devices and the like. The presentation tier may communicate with other tiers in the application such as the business logic tier and/or clinical trial or user/patient record data tier by accessing available components or web services provided by one or more of the other application tiers. The presentation tier may communicate with another tier to allow authorized users to access patient record data and/or database stored procedures, instructions, or protocols. The business logic tier can coordinate the application's functionality by processing commands, scheduling tests and evaluating data. The functionality of the business logic tier may be made accessible to other application tiers by, for example, the use of web services. The business logic tier may also provide the logic, instructions or security that can separate and distinguish clinical users from non-clinical users (e.g., administrative users). Where patient data is incorporated or accessed by the monitoring systems, a patient data record tier can hold the private patient records data and encapsulate such records from unapproved parties so as to comply with HIPAA or other privacy regulations. The patient records data tier can make data available through, for example, stored procedures, logic, instructions and the like accessible, for example, by web services.

As shown in FIG. 1, the system 10 can include at least one server 110 and a plurality of web clients 135, 135₁-135n (shown as 135₁, 135₂ for illustration only, with “n” being an integer number corresponding to the number of participating or registered users). Typically “n” is greater than 10, more typically, n is between 100-10,000, or even more, corresponding to the number of (registered) users U.

The at least one web server 110 can include a single web server as a control node (hub) or may include a plurality of servers (not shown). The system 10 can also include routers (not shown). For example, a router can coordinate privacy rules on data exchange or access. Where more than one server is used, different servers (and/or routers) may execute different tasks or may share tasks or portions of tasks. For example, the system 10 can include one or combinations of more than one of the following: a security management server, a registered participant/user directory server, a patient record management server, a scheduling server, and the like. The system 10 can include firewalls 115 and other secure connection and communication protocols. For Internet based applications, the server 110 and/or at least some of the associated web clients 135 can be configured to operate using SSL (Secure Sockets Layer) and a high level of encryption. Additional security functionality may also be provided. For example, incorporation of a communication protocol stack at the client and the server supporting SSL communications or Virtual Private Network (VPN) technology such as Internet Protocol Security Architecture (IPSec) may provide for secure communications to further assure a patient's privacy.

The system 10 can be provided using cloud computing which includes the provision of computational resources on demand via a computer network. The resources can be embodied as various infrastructure services (e.g., compute, storage, etc.) as well as applications, databases, file services, email, etc. In the traditional model of computing, both data and software are typically fully contained on the user's computer; in cloud computing, the user's computer may contain little software or data (perhaps an operating system and/or web browser), and may serve as little more than a display terminal for processes occurring on a network of external computers. A cloud computing service (or an aggregation of multiple cloud resources) may be generally referred to as the “Cloud”. Cloud storage may include a model of networked computer data storage where data is stored on multiple virtual servers, rather than being hosted on one or more dedicated servers.

The at least one server 110 can provide a centralized administration and management application. The at least one server 110 can be configured to provide session management, tracing and logging systems management, workload management and member services. The at least one server 110 can include or communicate with a plurality of databases including participant/user profiles, a security directory, routing security rules, and patient records. The at least one server 110 can include several sub-servers for integration into web systems, such as, but not limited to, a web application server (WAS) which may comprise an IBM WebSphere Application Server, a Directory Server such as an LDAP directory server, and may include an Anonymous Global Patient Identifier (AGPI) Server, a DB2 Server, and a Simple Mail Transfer Protocol (SMTP) Server. It is noted that although described herein as “servers” other suitable computer configurations may be used. The server 110 can be configured with web application functions that appear at portal sites 10p. The server 110 may comprise and/or be configured as a Web Sphere Business Integration (WBI) server. The at least one web server 110 can include a web-based administration application. The web application can be used to: allow a user to register as a participant, manage Access Control Lists (ACLs), logon using universal ID or password access, logoff, define profile preferences, search, approve clinical trial requests and the like.

The web clients 135 can be associated with different users and different user categories or types. Each category or type may have a different “privilege” or access level to actions or data associated with the systems 10. For example, the systems 10 can include clinician users, administrative users, and accounting users, each of which can have different access levels or restrictions to data and/or actions allowed by the system.

The system 10 can include a patient record database and/or server that can include electronic medical records (EMR) with privacy access restrictions that are in compliance with HIPPA rules due to the client-server operation and privilege defined access for different users.

The system 10 can be configured to provide at least one monitoring system APP. The system 10 can be configured to provide one APP for clinicians, one APP for routine monitoring of at-risk users U (for monitoring services or authorized representatives of a user U being monitored, such as a relative) and one system APP for users U. The patient APP can provide alerts of detected impairments or reduction in mobility or other triggered event (data fault, non-use of the support/IMU 20, low battery), patient therapy scheduling appointments, links to maps for a scheduled therapy location, automated voice, text message or other reminders of appointments, patient educational materials and the like to a defined device or devices, e.g., alert indicator 32 on support 30, smartphone, telephone, electronic tablet or notebook, local console 40, or other device.

The APP and/or the system 10 can also be configured to employ services related to online multimedia communications that may be provided by a third-party online multimedia communications service provider, which may be, e.g., a consumer videoconferencing service provider such as Skype, Microsoft Live Messenger, Yahoo! Messenger, America Online Instant Messenger, or Apple iChat that connects the user U to an authorized representative or caretaker, nurse or other person for ease of interaction.

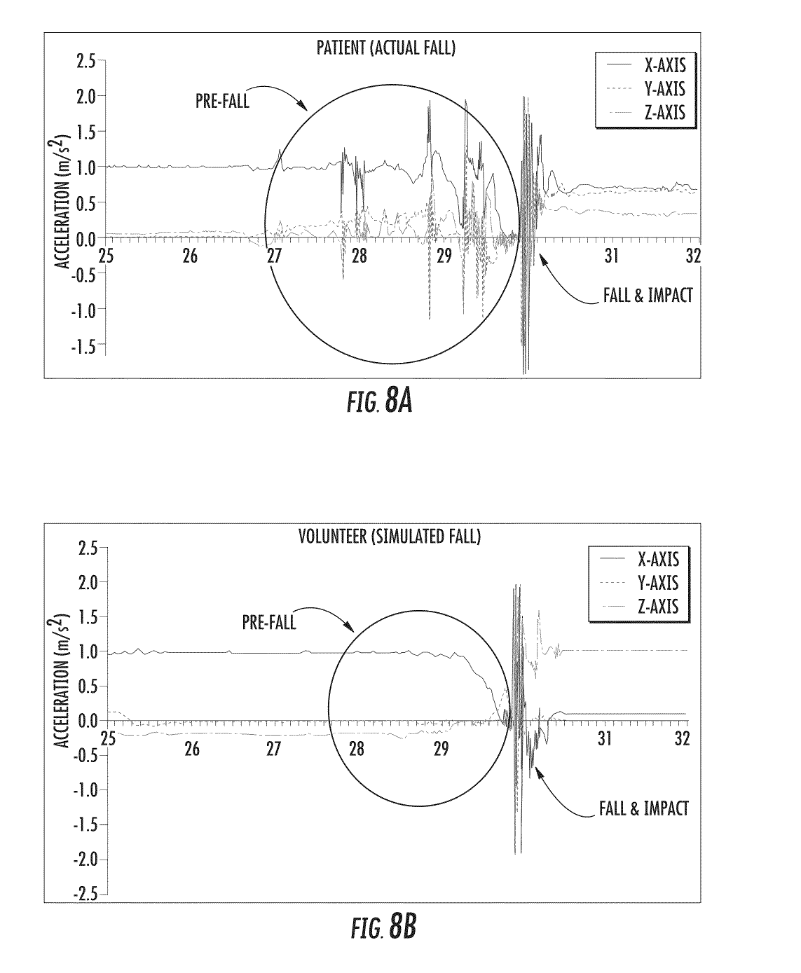

FIG. 8A is a graph of three acceleration waveform profiles showing pre-fall and actual fall accelerations for three orthogonal global system axes. FIG. 8B shows a corresponding waveform profiles that are based on a simulated fall by a volunteer. In some embodiments, the system 10 may be able to detect pre-fall activity signatures and/or a series of activity signatures demonstrating increasing activity instabilities which could trigger an alert to the user U to try to reduce the severity of a fall or to direct a user to seek medical treatment in order to prevent a future fall.

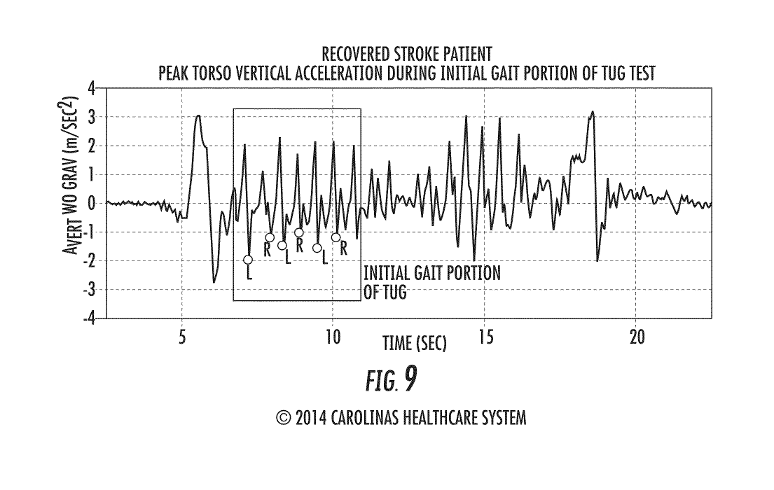

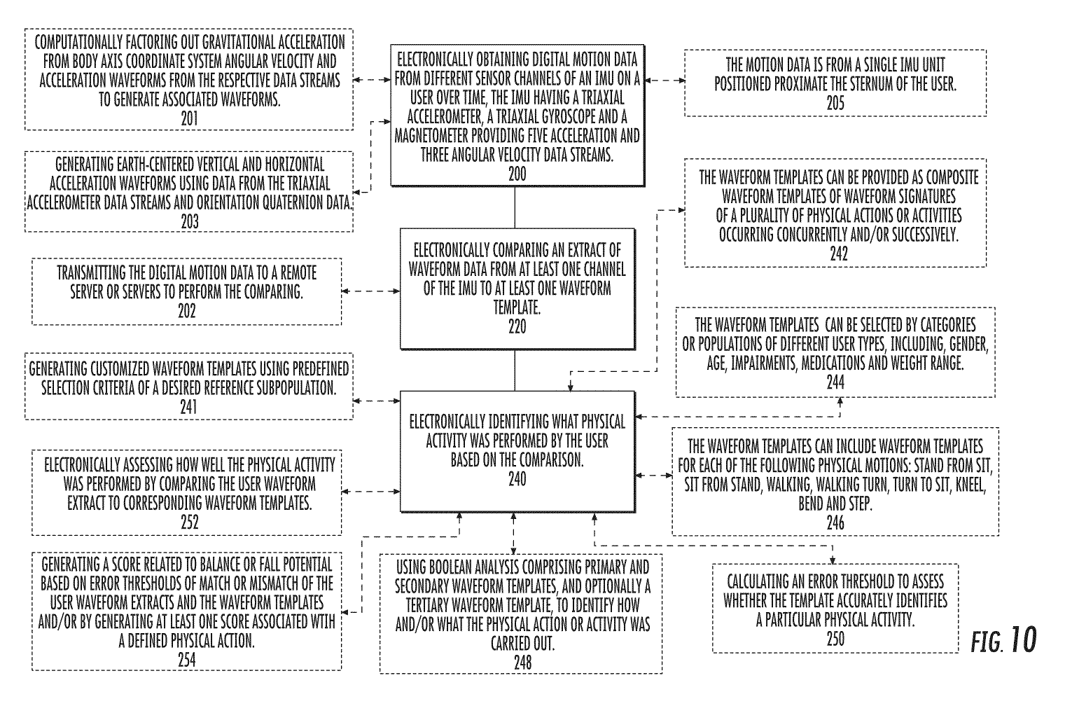

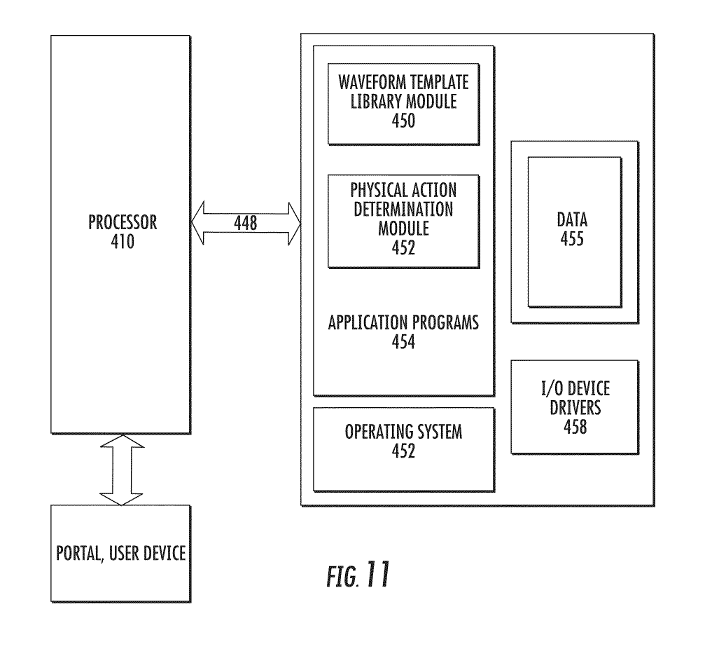

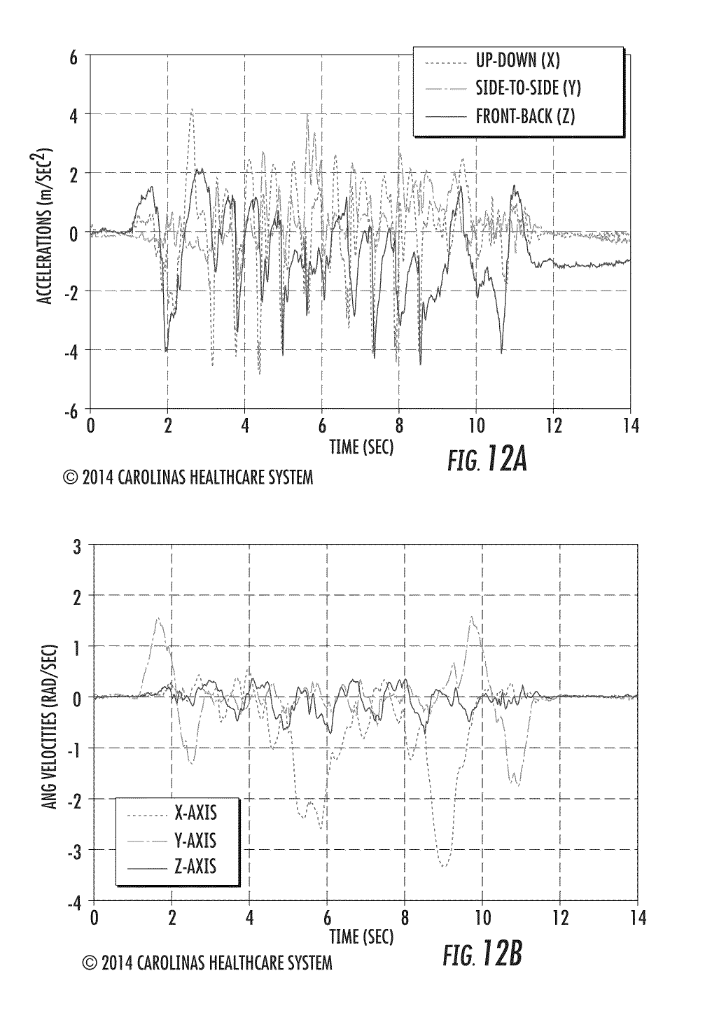

FIG. 9 illustrates a peak torso vertical acceleration activity waveform signature (m/sec²) during gait showing an irregular pattern associated with asymmetric movement of the right and left legs for a recovered stroke patient with slight impairment on the right side. The system 10 can still be used to monitor motion with this irregular condition and indeed can be configured to identify the irregular condition when monitoring for a “new” event or PA.

FIG. 10 illustrates exemplary actions that can be carried out to monitor a user. Electronically obtaining digital motion data from different sensor channels of an IMU on a user over time, the IMU having a triaxial accelerometer, a triaxial gyroscope and a magnetometer providing five acceleration and three angular velocity data streams (block 200). Electronically comparing an extract of waveform data from at least one channel of the IMU to at least one waveform template (block 220). Typically, a plurality of extracts from a plurality of different sensor channels are compared to one or a plurality of templates. Electronically identifying what physical activity was performed by the user based on the comparison (block 240).

The data from the data streams can be processed to computationally factor out gravitational acceleration from body axis coordinate system angular velocity and acceleration waveforms from the respective data streams to generate associated waveforms (block 201).

The waveform data can be used to generate earth-centered vertical and horizontal waveforms using data from some of the data streams and orientation quaternion data (block 203).

The digital motion data can be transmitted to a remote server or servers to perform the calculation and the comparing (block 202).

The motion data may be from a single IMU unit positioned proximate the sternum of the user (block 205).

The waveform templates can be provided as composite waveforms of a plurality of physical activities or actions occurring concurrently and/or successively (block 242).

The waveform templates can be selected by categories or populations of different user types, including, gender, age, impairments, medications and weight range (block 244).

The waveform templates can include waveform templates with waveform signatures for each of the following physical motions: stand from sit, sit from stand, walking, walking turn, turn to sit, kneel, bend and step (block 246).

An error threshold can be calculated to assess whether the template accurately identifies a particular physical activity (block 250).

Boolean analysis comprising primary and secondary waveform templates, and optionally a tertiary waveform template can be used to identify how the physical action or activity was carried out (block 248).

A user can be allowed to generate customized waveform profile templates using predefined selection criteria of a desired reference subpopulation (block 241).

How well the physical activity was performed can be electronically evaluated by comparing the user waveform extracts to corresponding waveform templates (block 252).

A score or scores related to balance or fall potential based on error thresholds of match or mismatch of the user waveform extracts and the waveform template can be generated s and/or by generating at least one score associated with a defined physical action (block 254).

As will be appreciated by one of skill in the art, embodiments of the invention may be embodied as a method, system, data processing system, or computer program product. Accordingly, the present invention may take the form of an entirely software embodiment or an embodiment combining software and hardware aspects, all generally referred to herein as a “circuit” or “module.” Furthermore, the present invention may take the form of a computer program product on a non-transient computer usable storage medium having computer usable program code embodied in the medium. Any suitable computer readable medium may be utilized including hard disks, CD-ROMs, optical storage devices, a transmission media such as those supporting the Internet or an intranet, or magnetic or other electronic storage devices.

Computer program code for carrying out operations of the present invention may be written in an object oriented programming language such as Java, Smalltalk, C# or C++. However, the computer program code for carrying out operations of the present invention may also be written in conventional procedural programming languages, such as the “C” programming language or in a visually oriented programming environment, such as Visual Basic.

Certain of the program code may execute entirely on one or more of a user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider). Typically, some program code executes on at least one web (hub) server and some may execute on at least one web client and with communication between the server(s) and clients using the Internet.

The invention is described in part below with reference to flowchart illustrations and/or block diagrams of methods, systems, computer program products and data and/or system architecture structures according to embodiments of the invention. It will be understood that each block of the illustrations, and/or combinations of blocks, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the block or blocks.

These computer program instructions may also be stored in a computer-readable memory or storage that can direct a computer or other programmable data processing apparatus to function in a particular manner, such that the instructions stored in the computer-readable memory or storage produce an article of manufacture including instruction means which implement the function/act specified in the block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the block or blocks.

The flowcharts and block diagrams of certain of the figures herein illustrate exemplary architecture, functionality, and operation of possible implementations of embodiments of the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order or two or more blocks may be combined, depending upon the functionality involved.

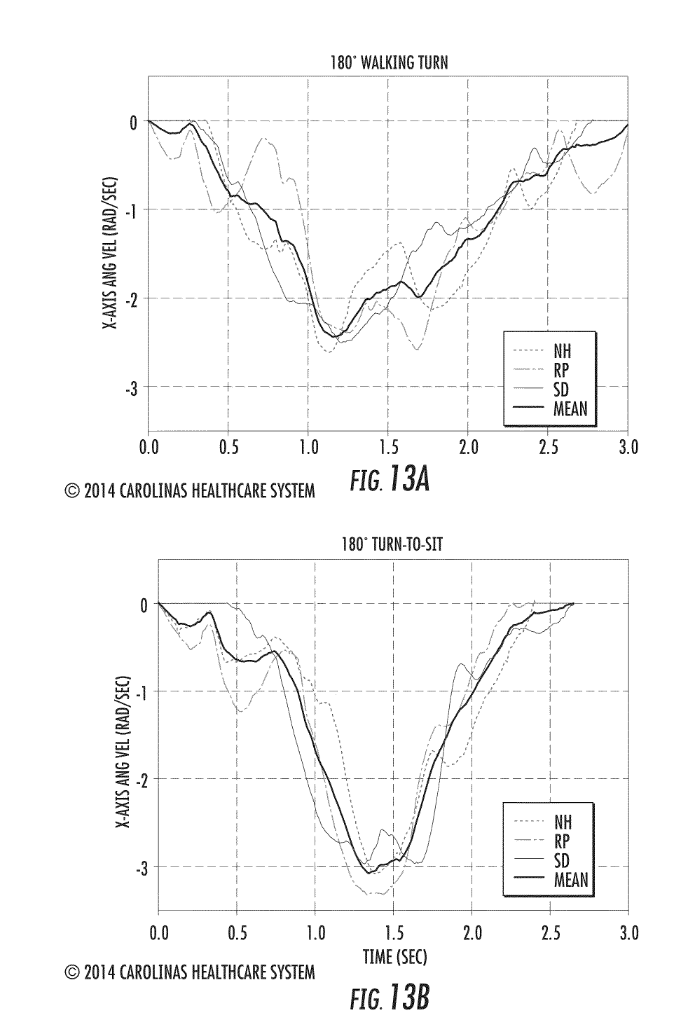

FIG. 11 is a schematic illustration of a circuit or data processing system 400 that can be used to provide the monitoring system 10. The circuits and/or data processing systems 400 may be incorporated in a digital signal processor in any suitable device or devices. As shown in FIG. 11, the processor 410 communicates with and/or is integral with clients or local user devices and with memory 414 via an address/data bus 448. The processor 410 can be any commercially available or custom microprocessor. The memory 414 is representative of the overall hierarchy of memory devices containing the software and data used to implement the functionality of the data processing system. The memory 414 can include, but is not limited to, the following types of devices: cache, ROM, PROM, EPROM, EEPROM, flash memory, SRAM, and DRAM.

FIG. 11 illustrates that the memory 414 may include several categories of software and data used in the data processing system: the operating system 449; the application programs 454; the input/output (I/O) device drivers 458; and data 455. The data 455 can include a library of defined waveform templates.

As will be appreciated by those of skill in the art, the operating systems 449 may be any operating system suitable for use with a data processing system, such as OS/2, AIX, or zOS from International Business Machines Corporation, Armonk, N.Y., Windows CE, Windows NT, Windows95, Windows98, Windows2000, WindowsXP, Windows Visa, Windows7, Windows CE or other Windows versions from Microsoft Corporation, Redmond, Wash., Palm OS, Symbian OS, Cisco IOS, VxWorks, Unix or Linux, Mac OS from Apple Computer, LabView, or proprietary operating systems.

The I/O device drivers 458 typically include software routines accessed through the operating system 449 by the application programs 45 to communicate with devices such as I/O data port(s), data storage 455 and certain memory 414 components. The application programs 45 are illustrative of the programs that implement the various features of the data processing system and can include at least one application, which supports operations according to embodiments of the present invention. Finally, the data 455 represents the static and dynamic data used by the application programs 45, the operating system 449, the I/O device drivers 458, and other software programs that may reside in the memory 414.

While the present invention is illustrated, for example, with reference to the Modules 450 and 452 being application programs in FIG. 11, as will be appreciated by those of skill in the art, other configurations may also be utilized while still benefiting from the teachings of the present invention. For example, the Modules and/or may also be incorporated into the operating system 449, the I/O device drivers 458 or other such logical division of the data processing system. Thus, the present invention should not be construed as limited to the configuration of FIG. 11 which is intended to encompass any configuration capable of carrying out the operations described herein. Further, one or more of modules, i.e., Modules 450, 452 can communicate with or be incorporated totally or partially in other components, such as separate or a single processor.

The I/O data port can be used to transfer information between the data processing system and another computer system or a network (e.g., the Internet) or to other devices controlled by the processor. These components may be conventional components such as those used in many conventional data processing systems, which may be configured in accordance with the present invention to operate as described herein.

Having now described the invention, the same will be illustrated with reference to certain examples, which are included herein for illustration purposes only, and which are not intended to be limiting of the invention.

### EXAMPLES

### Example 1

### EA Standardized Testing Using Template Matching Techniques

Standardized tests are commonly used to assess a patient's stability and potential for falling. Embodiments of the invention can be used to perform an electronically-augmented (EA) standardized test of a patient. One such test is a timed up and go (TUG) test, another is a five times sit-to-stand test (FTSTS) and a third is the ten meter walk. The standardized TUG test uses total time to perform the test as the only outcome measure. In addition to total time, the EATUG test can generate in excess of 20 outcome variables for an evaluation of how well a patient has performed all four of the basic mobility functions of the test.

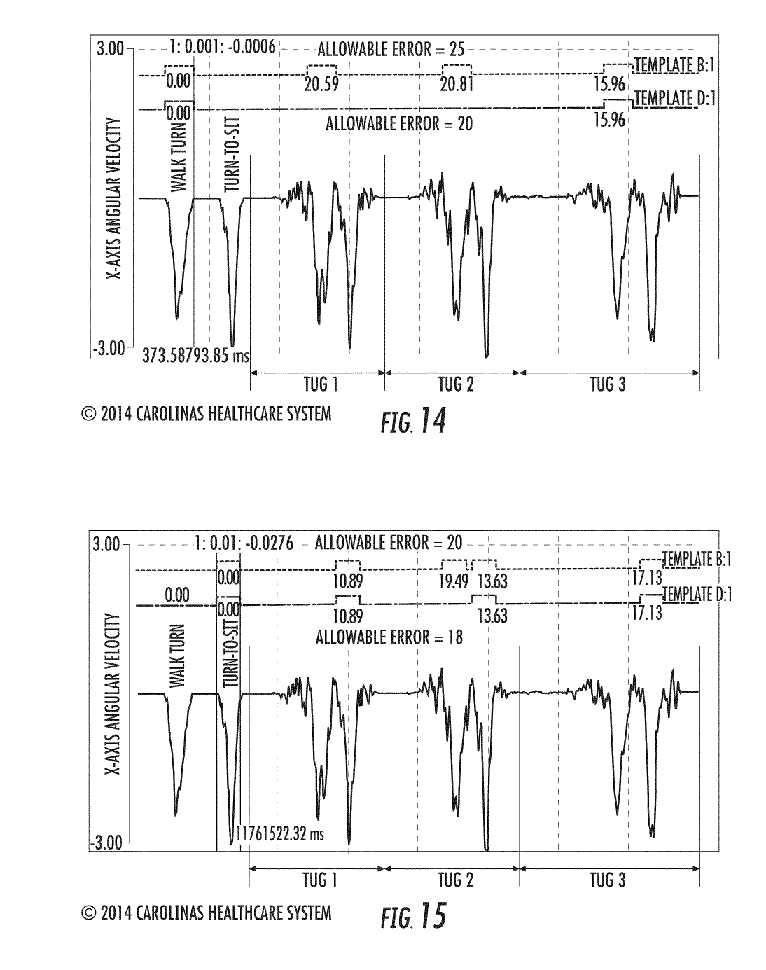

The standardized TUG test requires the patient to: stand up from a seated position, walk three meters, turn and walk back to the chair, turn and be seated. As previously stated, the total time to perform the test is typically used as the sole assessment parameter. In some embodiments, an electronically-augmented (EA) standardized test (shown here as a (EATUG) used an inertial measurement unit (IMU) that was affixed to the patient's torso at the top of the sternum using an elastic harness. The IMU monitors accelerations along three orthogonal body system axes. It also monitors angular velocities around these same axes. Orientation quaternion data is used to also calculate vertical and horizontal acceleration data for an earth centered axis system as previously described. FIGS. 12A, 12B and 12C show typical acceleration and angular velocity data collected during a typical EATUG test.

By way of example, template matching activity monitoring for the walking turn activity components of the test by evaluating angular velocity waveform profiles about the body system x-axis. FIGS. 13A and 13B show templates obtained by averaging turns from three different individuals. Both walking turns and the type of turn involved in sitting down (i.e. a turn-to-sit transfer-type turn) are shown. Next these two templates are applied to three concatenated body system x-axis waveform profiles for EATUG tests performed by three different individuals.

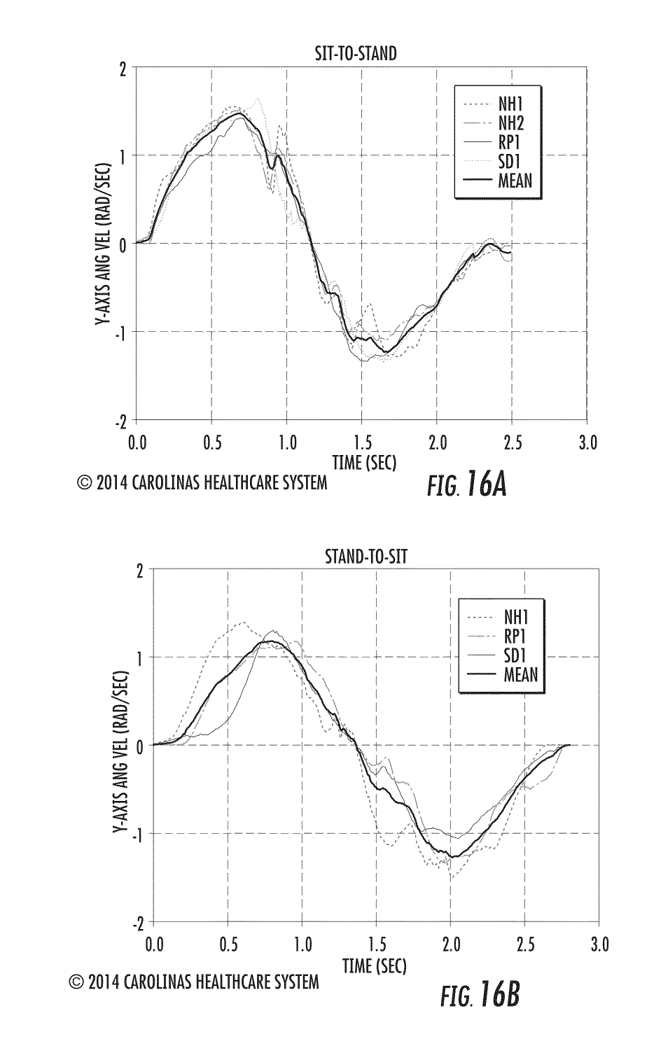

For demonstration purposes, FIG. 14 depicts a composite graph of waveforms constructed from the two separate x-axis turn templates (i.e. walk turn and turn-to-sit) and the three successive EATUG tests (i.e. TUG 1, TUG 2 and TUG 3) for that same x-axis angular velocity channel. In each EATUG test, the walking turn is performed first followed by the turn-to-sit at the end of the test. In FIG. 14, the walking turn template is first selected with an allowable error (AE) of 25. Note that all three of the walking turn events are positively identified and the actual errors are provided in order to assess “goodness-of-fit” (Template b:1, top) versus the mean template. Note, if the AE is set too low (i.e. AE=20) only one walking turn event is identified (Template d:1, top). In FIG. 15, the turn-to sit template is selected first with an AE of 20 (Template b:1, top) and then with an AE of 18 (Template d:1, top).

In the first case, with AE=20, one of the walking turns is incorrectly identified in TUG 2. This error in identification is corrected if the AE is lowered to 18. FIGS. 14 and 15 depict how strict template matching can be very sensitive to allowable error selection when only one template from one IMU data channel is used to identify an activity. Note that in FIGS. 14 and 15, two types of turns with subtle differences are used in the demonstration.

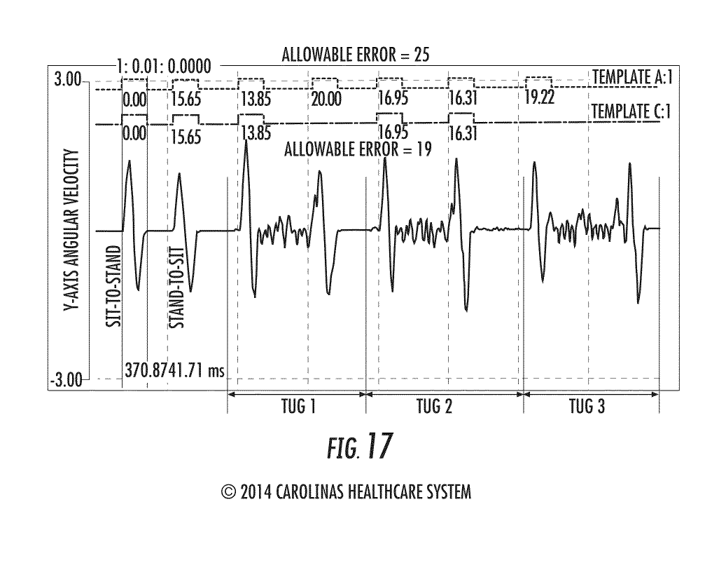

In some cases, however, two very different activities can demonstrate very similar waveform signatures and templates when only one data channel stream is examined. FIG. 16 shows torso flexion/extension (y-axis) angular velocity activity waveform signatures and averaged templates for sit-to-stand (FIG. 16A) and stand-to-sit (FIG. 16B) activities. Note, in this case that the stand-to-sit template is for simply sitting down, not for a turn-to-sit activity. The torso flexion and extension patterns are very similar for the two different activities. FIG. 17 shows the sit-to-stand template applied to the y-axis angular velocity channel data obtained during three EATUG test performed on different individuals. Once again, the sit-to-stand and stand-to-sit templates have been combined with data from the three EATUG tests as a continuous waveform for demonstration purposes. In FIG. 17, the stand-to-sit template is selected for matching v. the EATUG y-axis angular velocity data. At an AE of 25, all sit-to-stand and stand-to-sit waveform events are matched (Template a:1, top) except for the stand-to-sit event of TUG 3. At an AE of 19, event identification is improved but identification errors are still present (i.e. TUG 2 turn-to-sit improperly identified and TUG 3 sit-to-stand not identified).

FIG. 17, with TUG tests evaluated using AEs of 25 and 19, demonstrates how 100% accurate template matching is at times unachievable through AE reduction alone.

FIGS. 4A and 4B depict a more robust method of template matching which utilizes templates from different sensor channels collected simultaneously/concurrently to identify specific activities. In this case, torso vertical acceleration is examined concurrently with flexion/extension angular velocity for a single EATUG test. Note that while the initial (sit-to-stand) and final (stand-to-sit) patterns of the EATUG test look very similar (bottom graph) with regard to flexion/extension patterns, the concurrent vertical acceleration patterns are very different for the sit-to-stand and the stand-to-sit activities. While use of a single flexion/extension template matching technique may be adequate for activity detection, the necessity to template match multiple concurrent templates represents a Boolean approach for activity identification (i.e. if template 1 matches and template 2 matches, then the activity is accurately identified). Once an activity is properly identified, “goodness-of-fit” techniques, now using multiple templates, apply in the analysis.

The Boolean technique permits large allowable errors in template matching to be used in order to detect classes of activities while multiple concurrent templates are used to positively identify the specific activity being performed. Most activities of daily living related to mobility and gross stability functions involve relatively few primary motion patterns which combine to form more complex functions. The simple TUG test involves four mobility/stability activities (i.e. sit-to-stand, walking, turning and stand-to-sit). Representative activity templates for other activities such as stair climbing, retrieving an object from the floor, functional reach and different types of transfers are readily obtainable. The examples presented demonstrate how important gross body activities can be detected, identified and analyzed by a single torso-mounted IMU. A template library can be constructed with as few or as many activity templates as a particular monitoring task requires. A database with specific activity waveforms obtained from individuals of different ages or different medical conditions, however, can allow medical personnel to compose activity templates “on-the-fly” for groups of any age and/or medical condition for comparison with any individual currently being tested who meets those same criteria. This on-the-fly template construction approach also allows for comparisons of activity performance for a given individual versus groups of individuals selected using any combination of selection criteria.

### Example 2

### Medicine Titration/Side Effect Monitoring

The system 10 can be used for activity monitoring to assess and/or titrate the dosage of drugs with kinesiologic side effects that are administered to respective patients. The monitoring can be at a clinical site or at home. The monitoring can be for clinical trials or for normal therapeutic purposes.

### Example 3

### Rehabilitation/Physical Therapy

The system 10 can be used to electronically confirm a user is correctly performing a physical therapy exercise. The system 10 can generate an alert if the use is improper. The system can confirm that a user is performing the activity a recommended number of times per day or week. The system 10 can generate an alert, reminders or instructions if the user use is not following the recommended frequency or is performing exercises improperly.

### Example 4

### Video Game Interaction

The system 10 can be incorporated or integrated into a video game platform to allow a user to interact with the game based on the identified actions of the user.

### Example 5

### Automated Monitoring and Alerts

The system can be configured for short term or long term monitoring of ADLs in home or clinical settings to monitor users and provide feedback, alerts to caregivers or other assistants or alerts when detected physical activities diminish or performances changes.

Individuals can be monitored in the community and evaluated and scored for performance of kinesiological (motion-based) tests.

Another example is to monitor patients in hospitals, nursing facilities, rehabilitation facilities and assisted living facilities, etc. in an automated manner in order to reduce the patient monitoring load by staff. The technology is particularly useful for monitoring and assessing the various transfers common to such an environment.

### Example 6

### Long Duration ADL (Home) Monitoring

The system 10 may be used for remote patient monitoring to reduce unplanned hospitalization, ER visits, and improved patient self care.

### Example 7

### MEDICAL EVALUATION

The IMU 20 and support 30 may be provided “on-loan” for short-term kinematic evaluation pursuant to a doctor's order, similar to a portable cardio (Holter) monitor. The data can be obtained in near real time or post-monitoring.

### Example 8

### Physical Fitness/Exercise Self-Feedback

The monitoring system 10 may be configured for users during exercise such as at home or in a public or private gym or yoga facility, for example, for electronic feedback as to proper positioning and movement during a respective exercise which may help prevent injury due to improper use of equipment or improper form during such use.

### Example 9

### Use with GPS

Embodiments of the invention can be configured to employ navigation equations along with data from IMU sensors to determine the position of a user in a global coordinate system as a secondary analysis separate from the activity monitoring. If the IMU incorporates GPS technology, this capability could also be used for the same purpose as a secondary evaluation. This embodiment may be particularly suitable for users with impaired cognition/memory. The system 10 can be configured to have a “set-up mode” or a “learning mode” whereby an allowed perimeter of physical space is defined, e.g., a perimeter associated with a residence, house or yard, for example. This perimeter can be identified as a virtual fence so that an alert can be generated using the GPS evaluation if the user is determined to move outside this boundary.

### Example 10

### Evaluating Medication, Orthotics, Prosthetics or Implant Efficacy

Embodiments of the invention can be used to electronically evaluate physical action/activity of a patient to determine if activity kinematics change, improve or degrade during or after a therapy. For example, evaluation of kinematic changes of a user after receiving a spinal implant, a hip implant, or knee implant.

The evaluation can include comparing relative improvement of different users for different implants (from different manufacturers or different products). Government, medical institutions, insurance companies, or regulatory or decision making entities may use the information to assess whether to approve such an implant for use. The government may use such information during a 401K approval process. If a proposed new design does not indicate improvement over available implants, this may provide valuation data on whether to use within an institution. Older implant models are usually less expensive, if a new or newer model is not shown to provide an improvement, the increased cost for using the newer implant may be a deterrent.

The kinematic evaluation can be used to compare different orthotics or prosthetics to help a clinician decide which works better for a particular user.

The kinematic evaluation can be used in clinical trials of drugs to evaluate whether there is a negative, positive or no impact on a user participating in the trial.

### Example 11

### Animal Monitoring

The monitoring system can be configured to monitor animals during daily physical activities. The IMU can be placed on a harness on the animal or otherwise held for proper placement for suitable motion detection. The motion data can be used for training or to generate alerts when an animal exhibits an undesired physical activity such as voiding or eliminating in a home or when reacting to a “hot spot”. Other uses include monitoring one or more of: (i) after a surgical procedure for recovery (such as changes in gait after hip or spine surgery); (ii) during or after medication for efficacy or change or titration; or (iii) during or after physical therapy to assess any change in kinematics.

The animal monitoring can be configured to employ navigation equations from the IMU sensors to determine a position of a user in a global coordinate system. The GPS evaluation can be a secondary evaluation. This embodiment may be particularly suitable for “lost” pets.

The system 10 can be configured to have a “set-up mode” or a “learning mode” whereby an allowed perimeter of physical space is defined, e.g., a perimeter associated with a house or yard, for example. This perimeter can be identified as a virtual fence so that an alert can be generated if the animal is determined to move outside this boundary using the GPS evaluation.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A method of identifying a physical action and/or activity of a user in a free space environment comprising:
electronically obtaining digital data of physical activity and/or action of a user in free space from an inertial measurement unit (IMU) comprising a three axis accelerometer, a three axis gyroscope and a magnetometer, the IMU held proximate an upper torso of the user so as to be able to detect motion along and about a body axis coordinate system with three orthogonal axes, an up-down axis, a side-to-side axis and a front-to-back axis and provide orientation data of the body axis system with a global coordinate system;
electronically generating user waveforms of angular velocities and accelerations in the respective body axis system orthogonal axes over a first concurrent time period associated with a physical action and/or activity using the data obtained from the IMU;
electronically generating user waveforms of at least vertical acceleration in the global coordinate system in the first concurrent time period using the orientation data with acceleration data obtained from the IMU;
electronically comparing one or more waveform templates of angular velocities and/or accelerations associated with different defined actions and/or activities with one or more of the generated user waveforms; and
electronically identifying physical actions and/or activities of the user based on one or more waveform template matches from the electronic comparison without requiring a camera or other motion tracking systems in the environment.

2. The method of claim 1, wherein the obtaining step is carried out while the user performs random activities in free space without requiring a camera or other motion tracking systems in the environment.

3. The method of claim 1, wherein the obtaining step is carried out while the user performs assigned physical tasks or actions in free space without requiring a camera or other motion tracking systems in the environment.

4. The method of claim 1, wherein the predefined waveform templates are waveform templates of angular velocities and accelerations for specific defined activities comprising activity signatures from different users generated by calculating a mean or average of the activity signatures from the different users using IMU data obtained during each different user's performance of each specific defined activity.

5. The method of claim 1, further comprising electronically evaluating performance of the identified physical action and/or activity of the user based on error thresholds of matches from the IMU waveform(s) data to template(s) based on the comparison(s) without requiring a camera or other motion tracking systems in the environment.

6. The method of claim 1, wherein the IMU is a single IMU, and wherein the identification is carried out using the obtained data or motion data plus orientation data obtained only from the single IMU.

7. The method of claim 1, wherein the generated user waveforms include eight different waveforms over a concurrent time period from a respective eight data streams from the IMU, and wherein the electronic identification is carried out by comparing activity signatures in less than eight of the different waveforms, each template having one or more activity signatures for a defined action and/or activity.

8. The method of claim 1, wherein the generated user waveforms include eight different waveforms over a concurrent time period, and wherein the electronic identification is carried out by comparing activity signatures of between one and four of the different waveforms over a concurrent time period with waveform templates, each waveform template having one or more activity signatures for a defined action and/or activity.

9. The method of claim 1, wherein the generated user waveforms of angular velocities and accelerations with respect to the body axis system and corresponding waveform templates exclude gravity.

10. The method of claim 1, wherein the action or activity identification and performance evaluation comprises matching a primary waveform template and a secondary waveform template associated with a defined physical action or activity.

11. The method of claim 1, further comprising accepting user input to select one or more parameters of a reference population associated with waveform templates of angular velocities and accelerations for specific defined activities comprising activity signatures from different users during performance of each specific defined activity, and electronically generating a custom set of waveform templates based on the input to thereby allow different sets or categories of waveform templates for different users and/or different sets of users.

12. The method of claim 1, wherein the identifying comprises calculating an error threshold of at least one waveform template to at least one generated user waveform with an activity signature, and wherein, if the error threshold is below a defined value, then the physical action or activity is identified as the action or activity defined by the physical action or activity associated with a respective at least one waveform template.

13. The method of claim 1, wherein the identifying comprises electronically calculating an error threshold of a first waveform template to at least one generated user waveform with an activity signature, and wherein, if the error threshold is above a defined value, electronically comparing a second different selected waveform template to the at least one generated user waveform with the activity signature.

14. The method of claim 1, wherein the comparing comprises employing Boolean logic with different waveform templates using a defined pair or series of waveform templates that are used to distinguish a physical action or activity and/or characterize how a physical action or activity is carried out.

15. The method of claim 14, wherein the different waveform templates include a first waveform template of vertical acceleration in an up-down axis and a second waveform template of angular velocity in a side-side axis, wherein the first and second waveform templates are used to identify a stand to sit or sit to stand physical action.

16. The method of claim 1, further comprising providing at least one database of waveform templates, each with one or more activity signatures associated with defined physical actions and/or activities for the comparing and identifying steps, the database including one or more waveform templates for each of a plurality of the following: step up or step down, walk, sit to stand, stand to sit, reach, pick an object up, bend, stair climbing, stair descent, bed-chair transfer, and transfer from chair to toilet.

17. The method of claim 1, further comprising electronically identifying improvement or increasing kinematic impairment of the user by comparing generated user waveforms with activity signatures of the same physical activity or action at different times.

18. The method of claim 1, further comprising electronically assessing mobility status of a respective user by comparing different generated user waveform profiles with respective mean or average calculated waveform templates of the same physical action or activity generated using a reference population or subpopulation.

19. The method of claim 1, further comprising electronically evaluating a standardized physical activity and/or action test to assess a respective user's stability or fall risk using the generated user waveforms and the waveform templates, wherein the evaluation of the test includes evaluating a plurality of defined actions associated with a user undergoing the test that comprises at least one sit to stand and/or stand to sit physical action.

20. The method of claim 1, further comprising electronically evaluating how well a user is performing the identified action or activity by comparing maxima and/or minimas of an activity signature or signatures in one or more of the generated user waveforms to corresponding activity signatures in one or more waveform templates from a reference population.

21. The method of claim 1, further comprising electronically evaluating physical impairments, decreasing kinematic ability or improved kinematic ability from a drug, implant, prosthetic or orthotic of the user, using at least one of the generated user waveforms at one time and/or over time, wherein the waveform templates can comprise baseline waveforms of activity signatures of the user generated by monitoring the user when performing defined actions or activities.

22. The method of claim 1, further comprising electronically evaluating physical impairments, decreasing kinematic ability or improved kinematic ability from a drug, implant, prosthetic or orthotic of the user, using at least one of the generated user waveforms at one time and/or over time, wherein the waveform templates comprise activity signatures generated by calculating a mean or average of activity signatures from different users obtained during performance of each specific defined activity or action.

23. The method of claim 21, wherein the evaluating is carried out to evaluate influence or efficacy of a drug to allow a physician to proactively monitor for drug effects and/or to titrate a prescribed dose for a user.

24. The method of claim 1, further comprising electronically generating one or more scores of one or more physical activities or actions to assess fall risk and/or how normal or abnormal the physical activity of action was performed by a user.

25. A method for activity monitoring of users using a computer network, comprising:
providing a web-based service that provides an activity monitoring analysis module using at least one server that evaluates data obtained from a multi-channel IMU of different respective users, the IMU having at least eight data streams output to the web-based service including three accelerometer data streams, three angular velocity data streams and three magnetometer data streams, wherein the web-based activity monitoring analysis module is configured to identify a physical activity and/or action of respective users by comparing waveform templates in a database or electronic library to user waveforms with activity signatures associated with physical actions or activities of respective users generated from the data from respective IMUs without requiring a camera or other motion tracking systems in a user environment.

26. The method of claim 25, wherein the activity monitoring analysis module is also configured to evaluate how well the identified physical activity or action was performed relative to a reference population or relative to a baseline performance of the same physical activity or action.

27. The method of claim 25, wherein the activity monitoring analysis module is configured to identify the physical action or activity using template matching with a plurality of waveform templates from the database or library that have defined associated activity signatures to identify what physical activity is performed by a respective user using between 1-4 waveform templates.

28. The method of claim 25, wherein the data streams are used to generate a waveform extract of eight different user waveforms over a concurrent time period, wherein the identification is carried out by comparing activity signatures of between one and four of the different user waveforms over a concurrent time period with one or more activity signatures in one or more of the waveform templates, and wherein the waveform templates for some or all defined physical actions or activities include waveform templates of velocities and accelerations in a respective body axis system and a waveform template of vertical acceleration in a global coordinate system.

29. The method of claim 25, wherein the eight data streams include user waveforms of angular velocities and accelerations in a respective body axis system orthogonal axes over a first concurrent time period associated with physical actions and/or activities, and a waveform of vertical acceleration in a global coordinate system using orientation data with acceleration and angular velocity data in the body axis system obtained from the IMU.

30. A monitoring system comprising:
at least one web server in communication with a global computer network configured to provide a web-based service that hosts a monitoring analysis module that evaluates motion data obtained from an IMU comprising a three axis accelerometer, a three axis gyroscope and a magnetometer associated with respective different respective users to identify a physical activity carried out by respective users using data only from the IMU and a library or database of waveform templates without requiring a camera or other motion tracking systems in a free space environment.

31. The system of claim 30, wherein the monitoring analysis module is configured to identify how the physical activity was carried out using Boolean review with a defined hierarchy of selected waveform templates to compare to user waveforms generated from data from the IMU, and wherein the monitoring analysis module can optionally evaluate how well the physical activity was performed using the waveform templates generated relative to a reference population or a baseline performance by the user of the same physical activity.

32. The system of claim 30, wherein the analysis module is configured to obtain digital data of random physical activities and/or actions of respective users in free space from a single IMU for each user, the IMU held proximate an upper torso of the user so as to be able to detect motion along and about a body axis coordinate system with three orthogonal axes, an up-down axis, a side-to-side axis and a front-to-back axis and provide orientation data of the body axis system with a global coordinate system, wherein the analysis module is configured to (a) generate user waveforms of angular velocities and accelerations in the respective body axis system orthogonal axes over a first concurrent time period associated with physical actions and/or activities using the data obtained from the IMU; and (b) generate user waveforms of acceleration in the global coordinate system in the first concurrent time period using the orientation data with acceleration and angular velocity data in the body axis system obtained from the IMU.

33. The system of claim 30, wherein the library or database of waveform templates include waveform templates of angular velocities and/or accelerations with activity signatures associated with defined different actions or activities based on performance of the defined different actions or activities by different users with respective IMUs.

34. A computer program product, the computer program product comprising:
a non-transitory computer readable storage medium having computer readable program code, the computer-readable program code comprising:
computer readable program code configured to carry out the method of claim 1.

35. A system comprising:
at least one processor comprising computer program code that, when executed, causes the processor to carry out the operations of the method recited in claim 1.
